(19) United States
(12) Patent Application Publication
(10) Pub. No.: US 2023/0302646 A1
(43) Pub. Date: Sep. 28, 2023

(54) SYSTEMS AND METHODS FOR CONTROLLING AND ENHANCING MOVEMENT OF A SURGICAL ROBOTIC UNIT DURING SURGERY

(71) Applicant: Vicarious Surgical Inc., Waltham, MA (US)

(72) Inventors: Adam Sachs, Somerville, MA (US); Sammy Khalifa, Medford, MA (US); Fabrizio Santini, Arlington, MA (US); Spencer K. Howe, Scituate, MA (US); Daniel Allis, Boxford, MA (US); Maeve Devlin, East Boston, MA (US); Brian Talbot, Southborough, MA (US)

(21) Appl. No.: 18/126,224

(22) Filed: Mar. 24, 2023

Related U.S. Application Data
(60) Provisional application No. 63/339,179, filed on May 6, 2022.
(60) Provisional application No. 63/323,218, filed on Mar. 24, 2022.

(51) Int. Cl. B25J 9/16; A61B 34/30; A61B 90/00

(52) U.S. Cl. CPC B25J 9/1666; A61B 34/30; A61B 90/361; A61B 90/37; B25J 9/1697; A61B 2090/3983

(57) ABSTRACT

A surgical robotic system and a method of controlling a location of one or more robotic arms in a constrained space are disclosed herein. In some embodiments, the robotic system includes a robotic unit having robotic arms. The system further includes a camera assembly to generate a view an anatomical structure of a patient. The system further includes a controller configured to or programmed to define a constriction area defining safe movement of the robotic arms. The method includes defining safe movement of the robotic arms with respect to the constriction area.

### RELATED APPLICATIONS

This application claims priority to U.S. Provisional Application No. 63/323,218, filed Mar. 24, 2022, and U.S. Provisional Application No. 63/339,179, filed May 6, 2022, the entire contents of which are incorporated herein by reference.

### BACKGROUND

The present disclosure is directed to minimally invasive surgical devices and associated control methods, and is more specifically related to controlling robotic surgical systems that are inserted into a patent during surgery.

Since its inception in the early 1990s, the field of minimally invasive surgery has grown rapidly. While minimally invasive surgery vastly improves patient outcome, this improvement comes at a cost to the surgeon's ability to operate with precision and ease. During laparoscopy, the surgeon must insert laparoscopic instruments through a small incision in the patient's abdominal wall.

Existing robotic surgical devices attempted to solve these problems. Some existing robotic surgical devices replicate non-robotic laparoscopic surgery with additional degrees of freedom at the end of the instrument. However, even with many costly changes to the surgical procedure, existing robotic surgical devices have failed to provide improved patient outcome in the majority of procedures for which they are used. Additionally, existing robotic devices create increased separation between the surgeon and surgical end-effectors. This increased separation causes injuries resulting from the surgeon's misunderstanding of the motion and the force applied by the robotic device. Because the multiple degrees of freedom of many existing robotic devices are unfamiliar to a human operator, such as a surgeon, the surgeons typically undergo extensive training on robotic simulators before operating on a patient in order to minimize the likelihood of causing inadvertent injury to the patient.

To control existing robotic devices, a surgeon sits at a surgeon console or station and controls manipulators with his or her hands and feet. Additionally, robot cameras remain in a semi-fixed location, and are moved by a combined foot and hand motion from the surgeon. These semi-fixed cameras with limited fields of view result in difficulty visualizing the operating field.

### SUMMARY

The present disclosure is directed to systems and methods for controlling movement of a robotic unit during surgery. According to some embodiments, the system includes a controller configured to or programmed to execute instructions held in a memory to receive tissue contact constraint data and control a robotic unit having robotic arms in a manner to reduce possible damage to tissue in an area identified by the tissue contact constraint data. The system may further include a camera assembly to generate a view an anatomical structure of a patient and a display unit configured to display a view of the anatomical structure

According some embodiments, the present disclosure is directed to a method of controlling a location of one or more robotic arms in a constrained space. The method includes receiving tissue contact constraint data and controlling the one or more robotic arms in a manner to reduce possible damage to tissue in the area defined by the tissue contact constraint data.

According some embodiments, the present disclosure is directed to a system including a robotic arm assembly having robotic arms, a camera assembly, wherein the camera assembly generates image data of an internal region of a patient, and a controller. The controller is configured to or programmed to detect one or more markers in the image data, control movement of the robotic arms based on the one or more markers in the image data, and store the image data.

### BRIEF DESCRIPTION OF THE DRAWINGS

These and other features and advantages of the present disclosure will be more fully understood by reference to the following detailed description in conjunction with the attached drawings in which like reference numerals refer to like elements throughout the different views. The drawings illustrate principals of the disclosure and, although not to scale, show relative dimensions.

FIG. 1 schematically depicts an example surgical robotic system in accordance with some embodiments.

FIG. 2A is an example perspective view of a patient cart including a robotic support system coupled to a robotic subsystem of the surgical robotic system in accordance with some embodiments.

FIG. 2B is an example perspective view of an example operator console of a surgical robotic system of the present disclosure in accordance with some embodiments.

FIG. 3A schematically depicts an example side view of a surgical robotic system performing a surgery within an internal cavity of a subject in accordance with some embodiments.

FIG. 3B schematically depicts an example top view of the surgical robotic system performing the surgery within the internal cavity of the subject of FIG. 3A in accordance with some embodiments.

FIG. 4A is an example perspective view of a single robotic arm subsystem in accordance with some embodiments.

FIG. 4B is an example perspective side view of a single robotic arm of the single robotic arm subsystem of FIG. 4A in accordance with some embodiments.

FIG. 5 is an example perspective front view of a camera assembly and a robotic arm assembly in accordance with some embodiments.

FIG. 6 is a schematic representation of the controller of the present disclosure for providing control of movement of the robotic unit within a patient according to the teachings of the present disclosure.

FIGS. 7A-7D are illustrative representations of the types of markers that can be applied to the patient during the surgical procedure.

FIGS. 8A-8B are illustrative representations of the robotic arms automatically moving (e.g., snapping) to a specific marker when placed within a threshold distance thereof according to the teachings of the present disclosure.

FIG. 9A is a representation illustrating the constrained movement of the robotic arms relative to a selected plane according to the teachings of the present disclosure.

FIG. 9B is a representation illustrating the constrained movement of the robotic arms when disposed within a selected volume, during a surgical procedure, according to the teachings of the present disclosure.

FIG. 9C is a representation illustrating the system preventing the placement of the robotic arms in one or more selected volumes, during a surgical procedure, according to the teachings of the present disclosure.

FIG. 9D is a representation illustrating the system limiting movement of the robotic arms to a selected volume, during a surgical procedure, according to the teachings of the present disclosure.

FIG. 10 is a representation of a constriction plane for protecting tissue of the patient during surgery, according to the teachings of the present disclosure.

FIG. 11 schematically depicts a motion control system of a surgical robotic system, according to the teachings of the present disclosure.

FIG. 12 is a representation of a tissue area identified by contact with a robotic arm, according to the teachings of the present disclosure.

FIG. 13 is a flowchart representing a process for identifying a tissue area.

FIG. 14 is a flowchart representing a process for identifying a tissue area.

FIG. 15 is a representation of a defined depth allowance below a visceral floor, according to the teachings of the present disclosure.

FIG. 16A is a representation of operation of the robotic arms in a first side of a visceral floor, according to the teachings of the present disclosure.

FIG. 16B is a representation of operation of the robotic arms within a depth allowance of the visceral floor, according to the teachings of the present disclosure.

FIG. 16C is a representation of operation of the robotic arms within an approximate midpoint of the depth allowance and the visceral floor, according to the teachings of the present disclosure.

FIG. 16D is a representation of operation of the robotic arms at the depth allowance, according to the teachings of the present disclosure.

### DETAILED DESCRIPTION

The robotic system of the present disclosure assists the surgeon in controlling movement of a robotic unit during surgery in which the robotic unit is operable within a patient to minimize the risk of accidental injury to the patient during surgery. The surgeon defines an operable area with regards to tissue at the surgical site and the system implement one or more constraints on the arms of the robotic unit to prevent or impede progress of the arms outside of the constraints. The operable area or constraints may be defining with markers or by visual identification of portions of tissue.

In the following description, numerous specific details are set forth regarding the system and method of the present disclosure and the environment in which the system and method may operate, in order to provide a thorough understanding of the disclosed subject matter. It will be apparent to one skilled in the art, however, that the disclosed subject matter may be practiced without such specific details, and that certain features, which are well known in the art, are not described in detail in order to avoid complication and enhance clarity of the disclosed subject matter. In addition, it will be understood that any examples provided below are merely illustrative and are not to be construed in a limiting manner, and that it is contemplated by the present inventors that other systems, apparatuses, and/or methods can be employed to implement or complement the teachings of the present disclosure and are deemed to be within the scope of the present disclosure.

Notwithstanding advances in the field of robotic surgery, the possibility of accidentally injuring the patient when the surgical robotic unit is initially deployed in the patient or during the surgical procedure is a technical problem has not been adequately addressed. When operating, the surgeon can articulate the robot to access the entire interior region of the abdomen. Because of the extensive range of movement of the robotic unit, injuries can occur during insertion of the robotic unit or can occur “off-camera” where the surgical robotic unit accidentally injures tissue, an organ, or a blood vessel, outside of the field of view of the surgeon. For example, the surgical robotic unit may tear or pinch tissue within a surgical site such as the visceral floor. As such, injuries of this type may go undetected, which is highly problematic for the patient.

Described herein are systems and methods for solving the technical problem of accidentally injuring a patient. The system may define an area corresponding to tissue surrounding a surgical site, potentially including user input to identify the tissue. The system may then prevent movement or slow movement of robotic arms beyond the identified area, or beyond a depth allowance beyond the identified area, to prevent tissue damage. Additionally or alternatively, the system may provide indications to the user to inform the user of the position of the robotic arms relative to the identified area.

Although an exemplary embodiment is described as using a plurality of units to perform the exemplary process, it is understood that the exemplary processes may also be performed by one or a plurality of modules or units. Additionally, it is understood that the term controller, control unit, computing unit, and the like, refers to one or more hardware devices that include at least a memory and a processor and is specifically programmed to execute the processes described herein. The memory is configured to store the modules and the processor is specifically configured to execute the functions and operations associated with the modules to perform the one or more processes that are described herein.

Furthermore, control logic of the present disclosure may be embodied as non-transitory computer readable media on a computer readable medium containing executable program instructions executed by a processor, controller/control unit or the like. Examples of the computer readable mediums include, but are not limited to, ROM, RAM, compact disc (CD)-ROMs, magnetic tapes, floppy disks, flash drives, smart cards and optical data storage devices. The computer readable recording medium can also be distributed in network coupled computer systems so that the computer readable media is stored and executed in a distributed fashion, e.g., by a telematics server or a Controller Area Network (CAN). The control logic can also be implemented using application software that is stored in suitable storage and memory and processed using known processing devices. The control or computing unit as described herein can be implemented using any selected computer hardware that employs a processor, storage and memory.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The term “constriction area” as used herein is defined as a three-dimensional volume or a two-dimensional plane. The three-dimensional volume may be defined as a cube, cone, cylinder, or other three-dimensional shape or combination of shapes.

While the system and method of the present disclosure can be designed for use with one or more surgical robotic systems, the surgical robotic system of the present disclosure can also be employed in connection with any type of surgical system, including for example robotic surgical systems, straight-stick type surgical systems, virtual reality surgical systems, and laparoscopic systems. Additionally, the system of the present disclosure may be used in other non-surgical systems, where a user requires access to a myriad of information, while controlling a device or apparatus.

The robotic system of the present disclosure assists the surgeon in controlling movement of a robotic unit during surgery in which the robotic unit is operable within a patient. The control features of the present disclosure thus enable the surgeon to minimize the risk of accidental injury to the patient during surgery.

Like numerical identifiers are used throughout the figures to refer to the same elements.

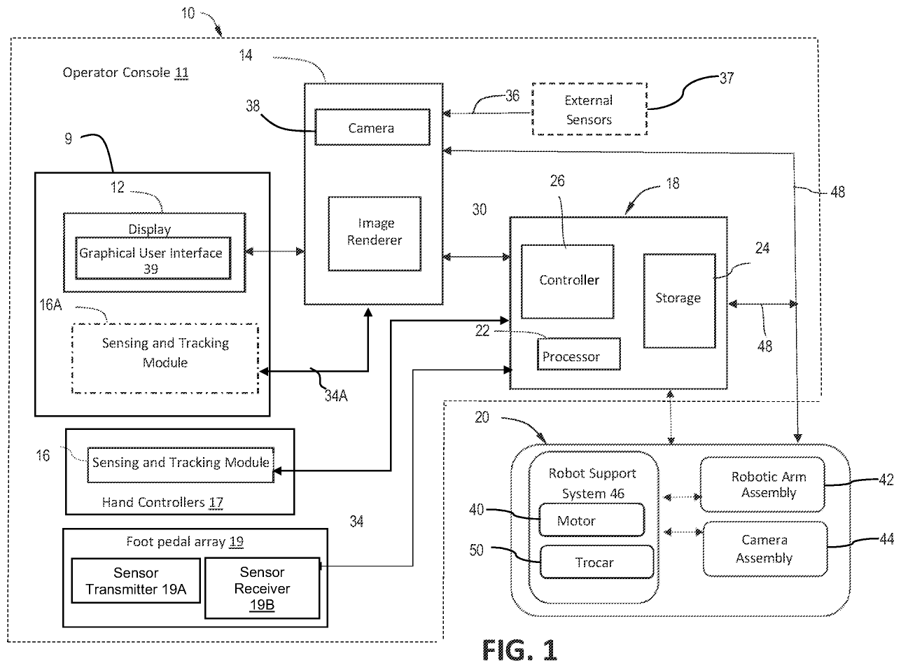

FIG. 1 is a schematic illustration of an example surgical robotic system 10 in which aspects of the present disclosure can be employed in accordance with some embodiments of the present disclosure. The surgical robotic system 10 includes an operator console 11 and a robotic subsystem 20 in accordance with some embodiments.

The surgical robotic system 10 of the present disclosure employs a robotic subsystem 20 that includes a robotic unit 50 that can be inserted into a patient via a trocar through a single incision point or site. The robotic unit 50 is small enough to be deployed in vivo at the surgical site and is sufficiently maneuverable when inserted within the patient to be able to move within the body to perform various surgical procedures at multiple different points or sites. The robotic unit 50 includes multiple separate robotic arms 42 that are deployable within the patient along different or separate axes. Further, a surgical camera assembly 44 can also be deployed along a separate axis and forms part of the robotic unit 50. Thus, the robotic unit 50 employs multiple different components, such as a pair of robotic arms and a surgical or robotic camera assembly, each of which are deployable along different axes and are separately manipulatable, maneuverable, and movable. Notably, the robotic unit 50 is not limited to the robotic arms and camera assembly described herein and additional components may be included in the robotic unit. The robotic arms and the camera assembly that are disposable along separate and manipulatable axes is referred to herein as the Split Arm (SA) architecture. The SA architecture is designed to simplify and increase efficiency of the insertion of robotic surgical instruments through a single trocar at a single insertion site, while concomitantly assisting with deployment of the surgical instruments into a surgical ready state as well as the subsequent removal of the surgical instruments through the trocar. By way of example, a surgical instrument can be inserted through the trocar to access and perform an operation in vivo in the abdominal cavity of a patient. In some embodiments, various surgical instruments may be utilized, including but not limited to robotic surgical instruments, as well as other surgical instruments known in the art.

The system and method disclosed herein can be incorporated and utilized with the robotic surgical device and associated system disclosed for example in U.S. Pat. No. 10,285,765 and in PCT patent application Serial No. PCT/US2020/39203, and/or with the camera assembly and system disclosed in United States Publication No. 2019/0076199, where the content and teachings of all of the foregoing patents, patent applications and publications are incorporated herein by reference. The robotic unit 50 can form part of the robotic subsystem 20, which in turn forms part of a surgical robotic system 10 that includes a surgeon or user workstation that includes appropriate sensors and displays, and a robot support system (RSS) or patient cart, for interacting with and supporting the robotic unit of the present disclosure. The robotic subsystem 20 can include, in one embodiment, a portion of the RSS, such as for example a drive unit and associated mechanical linkages, and the surgical robotic unit 50 can include one or more robotic arms and one or more camera assemblies. The surgical robotic unit 50 provides multiple degrees of freedom such that the robotic unit can be maneuvered within the patient into a single position or multiple different positions. In one embodiment, the robot support system can be directly mounted to a surgical table or to the floor or ceiling within an operating room. In another embodiment, the mounting is achieved by various fastening means, including but not limited to, clamps, screws, or a combination thereof. In still other embodiments, the structure may be free standing and portable or movable. The robot support system can mount the motor assembly that is coupled to the surgical robotic unit and can include gears, motors, drivetrains, electronics, and the like, for powering the components of the surgical robotic unit.

The robotic arms and the camera assembly are capable of multiple degrees of freedom of movement (e.g., at least seven degrees of freedom). According to one practice, when the robotic arms and the camera assembly are inserted into a patient through the trocar, they are capable of movement in at least the axial, yaw, pitch, and roll directions. The robotic arm assemblies are designed to incorporate and utilize a multi-degree of freedom of movement robotic arm with an end effector region mounted at a distal end thereof that corresponds to a wrist and hand area or joint of the user. In other embodiments, the working end (e.g., the end effector end) of the robotic arm is designed to incorporate and utilize other robotic surgical instruments, such as for example the surgical instruments set forth in U.S. Pat. No. 10,799,308, the contents of which are herein incorporated by reference.

The operator console 11 includes a display 12, an image computing module 14, which may be a three-dimensional (3D) computing module, hand controllers 17 having a sensing and tracking module 16, and a computing module 18. Additionally, the operator console 11 may include a foot pedal array 19 including a plurality of pedals. The image computing module 14 can include a graphical user interface 39. The graphical user interface 39, the controller 26 or the image renderer 30, or both, may render one or more images or one or more graphical user interface elements on the graphical user interface 39. For example, a pillar box associated with a mode of operating the surgical robotic system 10, or any of the various components of the surgical robotic system 10, can be rendered on the graphical user interface 39. Also live video footage captured by a camera assembly 44 can also be rendered by the controller 26 or the image renderer 30 on the graphical user interface 39.

The operator console 11 can include a visualization system 9 that includes a display 12 which may be any selected type of display for displaying information, images or video generated by the image computing module 14, the computing module 18, and/or the robotic subsystem 20. The display 12 can include or form part of, for example, a head-mounted display (HMD), an augmented reality (AR) display (e.g., an AR display, or AR glasses in combination with a screen or display), a screen or a display, a two-dimensional (2D) screen or display, a three-dimensional (3D) screen or display, and the like. The display 12 can also include an optional sensing and tracking module 16A. In some embodiments, the display 12 can include an image display for outputting an image from a camera assembly 44 of the robotic subsystem 20.

The hand controllers 17 are configured to sense a movement of the operator's hands and/or arms to manipulate the surgical robotic system 10. The hand controllers 17 can include the sensing and tracking module 16, circuitry, and/or other hardware. The sensing and tracking module 16 can include one or more sensors or detectors that sense movements of the operator's hands. In some embodiments, the one or more sensors or detectors that sense movements of the operator's hands are disposed in the hand controllers 17 that are grasped by or engaged by hands of the operator. In some embodiments, the one or more sensors or detectors that sense movements of the operator's hands are coupled to the hands and/or arms of the operator. For example, the sensors of the sensing and tracking module 16 can be coupled to a region of the hand and/or the arm, such as the fingers, the wrist region, the elbow region, and/or the shoulder region. Additional sensors can also be coupled to a head and/or neck region of the operator in some embodiments. In some embodiments, the sensing and tracking module 16 can be external and coupled to the hand controllers 17 via electricity components and/or mounting hardware. In some embodiments, the optional sensor and tracking module 16A may sense and track movement of one or more of an operator's head, of at least a portion of an operator's head, an operator's eyes or an operator's neck based, at least in part, on imaging of the operator in addition to or instead of by a sensor or sensors attached to the operator's body.

In some embodiments, the sensing and tracking module 16 can employ sensors coupled to the torso of the operator or any other body part. In some embodiments, the sensing and tracking module 16 can employ in addition to the sensors an Inertial Momentum Unit (IMU) having for example an accelerometer, gyroscope, magnetometer, and a motion processor. The addition of a magnetometer allows for reduction in sensor drift about a vertical axis. In some embodiments, the sensing and tracking module 16 also include sensors placed in surgical material such as gloves, surgical scrubs, or a surgical gown. The sensors can be reusable or disposable. In some embodiments, sensors can be disposed external of the operator, such as at fixed locations in a room, such as an operating room. The external sensors 37 can generate external data 36 that can be processed by the computing module 18 and hence employed by the surgical robotic system 10.

The sensors generate position and/or orientation data indicative of the position and/or orientation of the operator's hands and/or arms. The sensing and tracking modules 16 and/or 16A can be utilized to control movement (e.g., changing a position and/or an orientation) of the camera assembly 44 and robotic arms 42 of the robotic subsystem 20. The tracking and position data 34 generated by the sensing and tracking module 16 can be conveyed to the computing module 18 for processing by at least one processor 22.

The computing module 18 can determine or calculate, from the tracking and position data 34 and 34A, the position and/or orientation of the operator's hands or arms, and in some embodiments of the operator's head as well, and convey the tracking and position data 34 and 34A to the robotic subsystem 20. The tracking and position data 34, 34A can be processed by the processor 22 and can be stored for example in the storage 24. The tracking and position data 34 and 34A can also be used by the controller 26, which in response can generate control signals for controlling movement of the robotic arms 42 and/or the camera assembly 44. For example, the controller 26 can change a position and/or an orientation of at least a portion of the camera assembly 44, of at least a portion of the robotic arms 42, or both. In some embodiments, the controller 26 can also adjust the pan and tilt of the camera assembly 44 to follow the movement of the operator's head.

The robotic subsystem 20 can include a robot support system (RSS) 46 having a motor 40 and a trocar 50 or trocar mount, the robotic arms 42, and the camera assembly 44. The robotic arms 42 and the camera assembly 44 can form part of a single support axis robot system, such as that disclosed and described in U.S. Pat. No. 10,285,765, or can form part of a split arm (SA) architecture robot system, such as that disclosed and described in PCT Patent Application No. PCT/US2020/039203, both of which are incorporated herein by reference in their entirety.

The robotic subsystem 20 can employ multiple different robotic arms that are deployable along different or separate axes. In some embodiments, the camera assembly 44, which can employ multiple different camera elements, can also be deployed along a common separate axis. Thus, the surgical robotic system 10 can employ multiple different components, such as a pair of separate robotic arms and the camera assembly 44, which are deployable along different axes. In some embodiments, the robotic arms assembly 42 and the camera assembly 44 are separately manipulatable, maneuverable, and movable. The robotic subsystem 20, which includes the robotic arms 42 and the camera assembly 44, is disposable along separate manipulatable axes, and is referred to herein as an SA architecture. The SA architecture is designed to simplify and increase efficiency of the insertion of robotic surgical instruments through a single trocar at a single insertion point or site, while concomitantly assisting with deployment of the surgical instruments into a surgical ready state, as well as the subsequent removal of the surgical instruments through a trocar 50 as further described below.

The RSS 46 can include the motor 40 and the trocar 50 or a trocar mount. The RSS 46 can further include a support member that supports the motor 40 coupled to a distal end thereof. The motor 40 in turn can be coupled to the camera assembly 44 and to each of the robotic arms assembly 42. The support member can be configured and controlled to move linearly, or in any other selected direction or orientation, one or more components of the robotic subsystem 20. In some embodiments, the RSS 46 can be free standing. In some embodiments, the RSS 46 can include the motor 40 that is coupled to the robotic subsystem 20 at one end and to an adjustable support member or element at an opposed end.

The motor 40 can receive the control signals generated by the controller 26. The motor 40 can include gears, one or more motors, drivetrains, electronics, and the like, for powering and driving the robotic arms 42 and the cameras assembly 44 separately or together. The motor 40 can also provide mechanical power, electrical power, mechanical communication, and electrical communication to the robotic arms 42, the camera assembly 44, and/or other components of the RSS 46 and robotic subsystem 20. The motor 40 can be controlled by the computing module 18. The motor 40 can thus generate signals for controlling one or more motors that in turn can control and drive the robotic arms 42, including for example the position and orientation of each robot joint of each robotic arm, as well as the camera assembly 44. The motor 40 can further provide for a translational or linear degree of freedom that is first utilized to insert and remove each component of the robotic subsystem 20 through a trocar 50. The motor 40 can also be employed to adjust the inserted depth of each robotic arm 42 when inserted into the patient 100 through the trocar 50.

The trocar 50 is a medical device that can be made up of an awl (which may be a metal or plastic sharpened or non-bladed tip), a cannula (essentially a hollow tube), and a seal in some embodiments. The trocar 50 can be used to place at least a portion of the robotic subsystem 20 in an interior cavity of a subject (e.g., a patient) and can withdraw gas and/or fluid from a body cavity. The robotic subsystem 20 can be inserted through the trocar 50 to access and perform an operation in vivo in a body cavity of a patient. In some embodiments, the robotic subsystem 20 can be supported, at least in part, by the trocar 50 or a trocar mount with multiple degrees of freedom such that the robotic arms 42 and the camera assembly 44 can be maneuvered within the patient into a single position or multiple different positions. In some embodiments, the robotic arms 42 and camera assembly 44 can be moved with respect to the trocar 50 or a trocar mount with multiple different degrees of freedom such that the robotic arms 42 and the camera assembly 44 can be maneuvered within the patient into a single position or multiple different positions.

In some embodiments, the RSS 46 can further include an optional controller for processing input data from one or more of the system components (e.g., the display 12, the sensing and tracking module 16, the robotic arms 42, the camera assembly 44, and the like), and for generating control signals in response thereto. The motor 40 can also include a storage element for storing data in some embodiments.

The robotic arms 42 can be controlled to follow the scaled-down movement or motion of the operator's arms and/or hands as sensed by the associated sensors in some embodiments and in some modes of operation. The robotic arms 42 include a first robotic arm including a first end effector at distal end of the first robotic arm, and a second robotic arm including a second end effector disposed at a distal end of the second robotic arm. In some embodiments, the robotic arms 42 can have portions or regions that can be associated with movements associated with the shoulder, elbow, and wrist joints as well as the fingers of the operator. For example, the robotic elbow joint can follow the position and orientation of the human elbow, and the robotic wrist joint can follow the position and orientation of the human wrist. The robotic arms 42 can also have associated therewith end regions that can terminate in end-effectors that follow the movement of one or more fingers of the operator in some embodiments, such as for example the index finger as the user pinches together the index finger and thumb. In some embodiments, while the robotic arms 42 may follow movement of the arms of the operator in some modes of control while a virtual chest of the robotic arms assembly may remain stationary (e.g., in an instrument control mode). In some embodiments, the position and orientation of the torso of the operator are subtracted from the position and orientation of the operator's arms and/or hands. This subtraction allows the operator to move his or her torso without the robotic arms moving. Further disclosure control of movement of individual arms of a robotic arm assembly is provided in International Patent Application Publications WO 2022/094000 A1 and WO 2021/231402 A1, each of which is incorporated by reference herein in its entirety.

The camera assembly 44 is configured to provide the operator with image data 48, such as for example a live video feed of an operation or surgical site, as well as enable the operator to actuate and control the cameras forming part of the camera assembly 44. In some embodiments, the camera assembly 44 can include one or more cameras (e.g., a pair of cameras), the optical axes of which are axially spaced apart by a selected distance, known as the inter-camera distance, to provide a stereoscopic view or image of the surgical site. In some embodiments, the operator can control the movement of the cameras via movement of the hands via sensors coupled to the hands of the operator or via hand controllers 17 grasped or held by hands of the operator, thus enabling the operator to obtain a desired view of an operation site in an intuitive and natural manner. In some embodiments, the operator can additionally control the movement of the camera via movement of the operator's head. The camera assembly 44 is movable in multiple directions, including for example in yaw, pitch and roll directions relative to a direction of view. In some embodiments, the components of the stereoscopic cameras can be configured to provide a user experience that feels natural and comfortable. In some embodiments, the interaxial distance between the cameras can be modified to adjust the depth of the operation site perceived by the operator.

The image or video data 48 generated by the camera assembly 44 can be displayed on the display 12. In embodiments in which the display 12 includes an HMD, the display can include the built-in sensing and tracking module 16A that obtains raw orientation data for the yaw, pitch and roll directions of the HMD as well as positional data in Cartesian space (x, y, z) of the HMD. In some embodiments, positional and orientation data regarding an operator's head may be provided via a separate head-tracking module. In some embodiments, the sensing and tracking module 16A may be used to provide supplementary position and orientation tracking data of the display in lieu of or in addition to the built-in tracking system of the HMD. In some embodiments, no head tracking of the operator is used or employed. In some embodiments, images of the operator may be used by the sensing and tracking module 16A for tracking at least a portion of the operator's head.

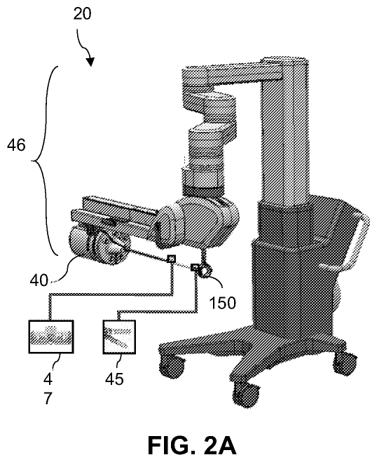

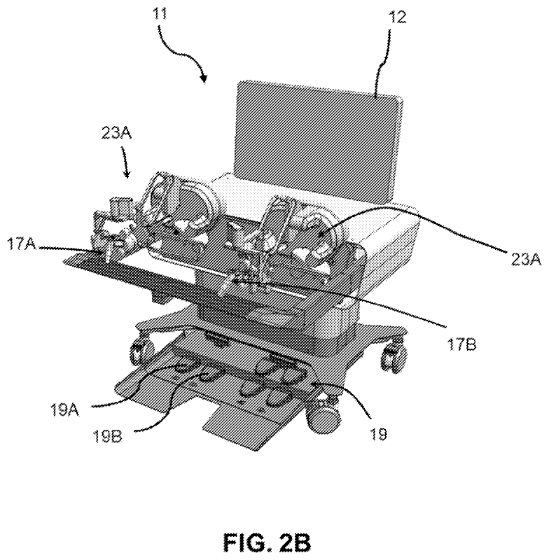

FIG. 2A depicts an example robotic arms assembly 20, which is also referred to herein as a robotic subsystem, of a surgical robotic system 10 incorporated into or mounted onto a mobile patient cart in accordance with some embodiments. In some embodiments, the robotic arms assembly 20 includes the RSS 46, which, in turn includes the motor 40, the robotic arm assembly 42 having end-effectors 45, the camera assembly 44 having one or more cameras 47, and may also include the trocar 50 or a trocar mount.

FIG. 2B depicts an example of an operator console 11 of the surgical robotic system 10 of the present disclosure in accordance with some embodiments. The operator console 11 includes a display 12, hand controllers 17, and also includes one or more additional controllers, such as a foot pedal array 19 for control of the robotic arms 42, for control of the camera assembly 44, and for control of other aspects of the system.

FIG. 2B also depicts the left hand controller subsystem 23A and the right hand controller subsystem 23B of the operator console. The left hand controller subsystem 23A includes and supports the left hand controller 17A and the right hand controller subsystem 23B includes and supports the right hand controller 17B. In some embodiments, the left hand controller subsystem 23A may releasably connect to or engage the left hand controller 17A, and right hand controller subsystem 23B may releasably connect to or engage the right hand controller 17A. In some embodiments, the connections may be both physical and electronic so that the left hand controller subsystem 23A and the right hand controller subsystem 23B may receive signals from the left hand controller 17A and the right hand controller 17B, respectively, including signals that convey inputs received from a user selection on a button or touch input device of the left hand controller 17A or the right hand controller 17B.

Each of the left hand controller subsystem 23A and the right hand controller subsystem 23B may include components that enable a range of motion of the respective left hand controller 17A and right hand controller 17B, so that the left hand controller 17A and right hand controller 17B may be translated or displaced in three dimensions and may additionally move in the roll, pitch, and yaw directions. Additionally, each of the left hand controller subsystem 23A and the right hand controller subsystem 23B may register movement of the respective left hand controller 17A and right hand controller 17B in each of the forgoing directions and may send a signal providing such movement information to a processor (not shown) of the surgical robotic system.

In some embodiments, each of the left hand controller subsystem 23A and the right hand controller subsystem 23B may be configured to receive and connect to or engage different hand controllers (not shown). For example, hand controllers with different configurations of buttons and touch input devices may be provided. Additionally, hand controllers with a different shape may be provided. The hand controllers may be selected for compatibility with a particular surgical robotic system or a particular surgical robotic procedure or selected based upon preference of an operator with respect to the buttons and input devices or with respect to the shape of the hand controller in order to provide greater comfort and ease for the operator.

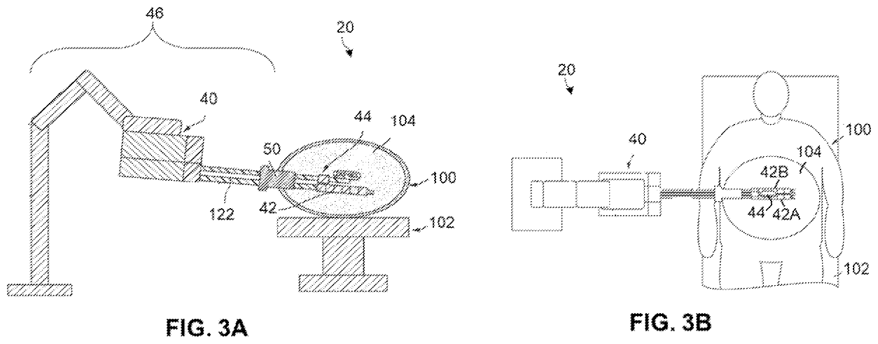

FIG. 3A schematically depicts a side view of the surgical robotic system 10 performing a surgery within an internal cavity 104 of a subject 100 in accordance with some embodiments and for some surgical procedures. FIG. 3B schematically depicts a top view of the surgical robotic system 10 performing the surgery within the internal cavity 104 of the subject 100. The subject 100 (e.g., a patient) is placed on an operation table 102 (e.g., a surgical table 102). In some embodiments, and for some surgical procedures, an incision is made in the patient 100 to gain access to the internal cavity 104. The trocar 50 is then inserted into the patient 100 at a selected location to provide access to the internal cavity 104 or operation site. The RSS 46 can then be maneuvered into position over the patient 100 and the trocar 50. In some embodiments, the RSS 46 includes a trocar mount that attaches to the trocar 50. The robotic arms assembly 20 can be coupled to the motor 40 and at least a portion of the robotic arms assembly can be inserted into the trocar 50 and hence into the internal cavity 104 of the patient 100. For example, the camera assembly 44 and the robotic arm assembly 42 can be inserted individually and sequentially into the patient 100 through the trocar 50. Although the camera assembly and the robotic arm assembly may include some portions that remain external to the subject's body in use, references to insertion of the robotic arm assembly 42 and/or the camera assembly into an internal cavity of a subject and disposing the robotic arm assembly 42 and/or the camera assembly 44 in the internal cavity of the subject are referring to the portions of the robotic arm assembly 42 and the camera assembly 44 that are intended to be in the internal cavity of the subject during use. The sequential insertion method has the advantage of supporting smaller trocars and thus smaller incisions can be made in the patient 100, thus reducing the trauma experienced by the patient 100. In some embodiments, the camera assembly 44 and the robotic arm assembly 42 can be inserted in any order or in a specific order. In some embodiments, the camera assembly 44 can be followed by a first robotic arm of the robotic arm assembly 42 and then followed by a second robotic arm of the robotic arm assembly 42 all of which can be inserted into the trocar 50 and hence into the internal cavity 104. Once inserted into the patient 100, the RSS 46 can move the robotic arm assembly 42 and the camera assembly 44 to an operation site manually or automatically controlled by the operator console 11.

Further disclosure regarding control of movement of individual arms of a robotic arm assembly is provided in International Patent Application Publications WO 2022/094000 A1 and WO 2021/231402 A1, each of which is incorporated by reference herein in its entirety.

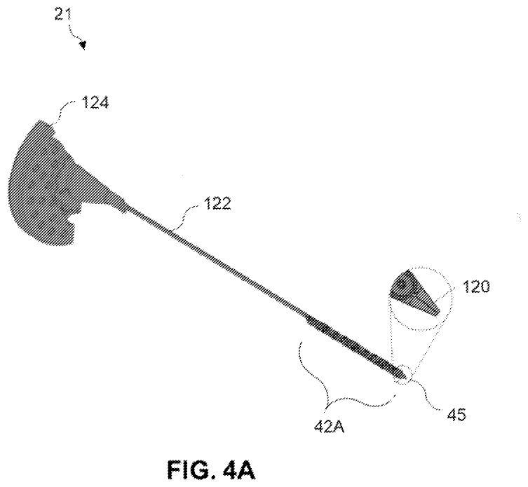

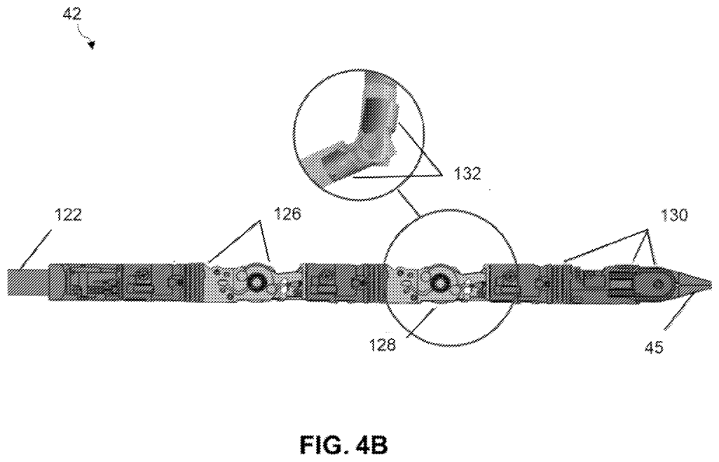

FIG. 4A is a perspective view of a robotic arm subassembly 21 in accordance with some embodiments. The robotic arm subassembly 21 includes a robotic arm 42A, the end-effector 45 having an instrument tip 120 (e.g., monopolar scissors, needle driver/holder, bipolar grasper, or any other appropriate tool), a shaft 122 supporting the robotic arm 42A. A distal end of the shaft 122 is coupled to the robotic arm 42A, and a proximal end of the shaft 122 is coupled to a housing 124 of the motor 40 (as shown in FIG. 2A). At least a portion of the shaft 122 can be external to the internal cavity 104 (as shown in FIGS. 3A and 3B). At least a portion of the shaft 122 can be inserted into the internal cavity 104 (as shown in FIGS. 3A and 3B).

FIG. 4B is a side view of the robotic arm assembly 42. The robotic arm assembly 42 includes a virtual shoulder 126, a virtual elbow 128 having position sensors 132 (e.g., capacitive proximity sensors), a virtual wrist 130, and the end-effector 45 in accordance with some embodiments. The virtual shoulder 126, the virtual elbow 128, the virtual wrist 130 can include a series of hinge and rotary joints to provide each arm with positionable, seven degrees of freedom, along with one additional grasping degree of freedom for the end-effector 45 in some embodiments.

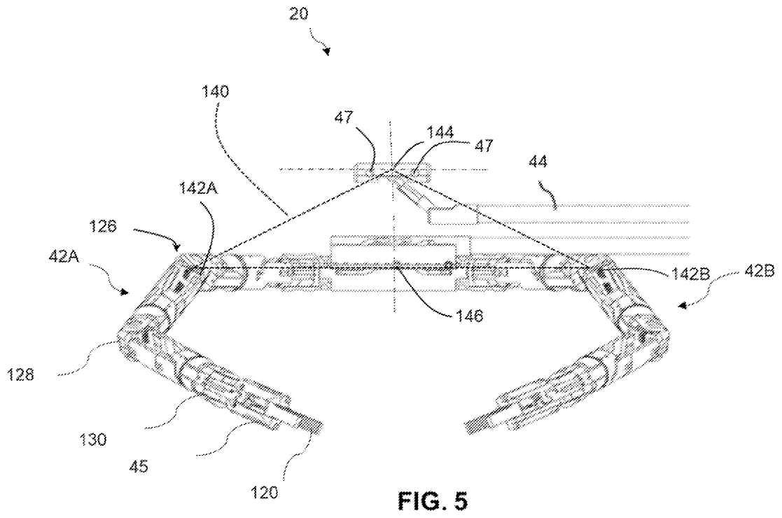

FIG. 5 illustrates a perspective front view of a portion of the robotic arms assembly 20 configured for insertion into an internal body cavity of a patient. The robotic arms assembly 20 includes a robotic arm 42A and a robotic arm 42B. The two robotic arms 42A and 42B can define, or at least partially define, a virtual chest 140 of the robotic arms assembly 20 in some embodiments. In some embodiments, the virtual chest 140 (depicted as a triangle with dotted lines) can be defined by a chest plane extending between a first pivot point 142A of a most proximal joint of the robotic arm 42A (e.g., a shoulder joint 126), a second pivot point 142B of a most proximal joint of the robotic arm 42B, and a camera imaging center point 144 of the camera(s) 47. A pivot center 146 of the virtual chest 140 lies in the middle of the virtual chest.

In some embodiments, sensors in one or both of the robotic arm 42A and the robotic arm 42B can be used by the system to determine a change in location in three-dimensional space of at least a portion of the robotic arm. In some embodiments, sensors in one or both of the first robotic arm and second robotic arm can be used by the system to determine a location in three-dimensional space of at least a portion of one robotic arm relative to a location in three-dimensional space of at least a portion of the other robotic arm.

In some embodiments, a camera assembly 44 is configured to obtain images from which the system can determine relative locations in three-dimensional space. For example, the camera assembly may include multiple cameras, at least two of which are laterally displaced from each other relative to an imaging axis, and the system may be configured to determine a distance to features within the internal body cavity. Further disclosure regarding a surgical robotic system including camera assembly and associated system for determining a distance to features may be found in International Patent Application Publication No. WO 2021/159409, entitled “System and Method for Determining Depth Perception In Vivo in a Surgical Robotic System,” and published Aug. 12, 2021, which is incorporated by reference herein in its entirety. Information about the distance to features and information regarding optical properties of the cameras may be used by a system to determine relative locations in three-dimensional space.

Hand controllers for a surgical robotic system as described herein can be employed with any of the surgical robotic systems described above or any other suitable surgical robotic system. Further, some embodiments of hand controllers described herein may be employed with semi-robotic endoscopic surgical systems that are only robotic in part.

As explained above, controllers for a surgical robotic system may desirably feature sufficient inputs to provide control of the system, an ergonomic design and “natural” feel in use.

In some embodiments described herein, reference is made to a left hand controller and a corresponding left robotic arm, which may be a first robotic arm, and to a right hand controller and a corresponding right robotic arm, which may be a second robotic arm. In some embodiments, a robotic arm considered a left robotic arm and a robotic arm considered a right robotic arm may change due a configuration of the robotic arms and the camera assembly being adjusted such that the second robotic arm corresponds to a left robotic arm with respect to a view provided by the camera assembly and the first robotic arm corresponds to a right robotic arm with respect view provided by the camera assembly. In some embodiments, the surgical robotic system changes which robotic arm is identified as corresponding to the left hand controller and which robotic arm is identified as corresponding to the right hand controller during use. In some embodiments, at least one hand controller includes one or more operator input devices to provide one or more inputs for additional control of a robotic assembly. In some embodiments, the one or more operator input devices receive one or more operators inputs for at least one of: engaging a scanning mode, resetting a camera assembly orientation and position to a align a view of the camera assembly to the instrument tips and to the chest; displaying a menu, traversing a menu or highlighting options or items for selection and selecting an item or option, selecting and adjusting an elbow position, and engaging a clutch associated with an individual hand controller.

In some embodiments, additional functions may be accessed via the menu, for example, selecting a level of a grasper force (e.g., high/low), selecting an insertion mode, an extraction mode, or an exchange mode, adjusting a focus, lighting, or a gain, camera cleaning, motion scaling, rotation of camera to enable looking down, etc.

As described herein, the robotic unit 50 can be inserted within the patient through a trocar. The robotic unit 50 can be employed by the surgeon to place one or more markers within the patient according to known techniques. For example, the markers can be applied using a biocompatible ink pen or the markers can be a passive object such as a QR code or an active object. The surgical robotic system 10 can then detect or track the markers within the image data with the detection unit 60. Markers may also be configured to emit an RF or electromagnetic signal to be detected by the detection unit 60. The detection unit 60 may be configured to identify specific structure, such as different marker types, and may be configured to utilize one or more known image detection techniques, such as by using sensors or detectors forming part of a computer vision system or by employing image disparity techniques using the camera assembly 44. According to one embodiment, the detection unit 60 may be configured to identify the markers in the captured image data 44, thus allowing the system 10 to detect and track the markers. By identifying and tracking the markers, the system allows the surgeon to accurately identify and navigate the robotic unit through the vagaries of the patient's anatomy.

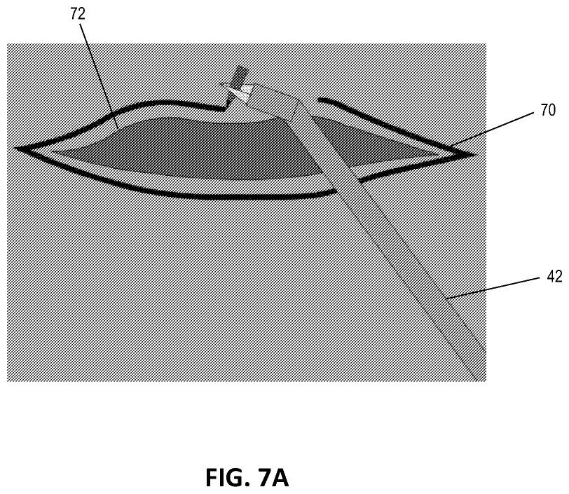

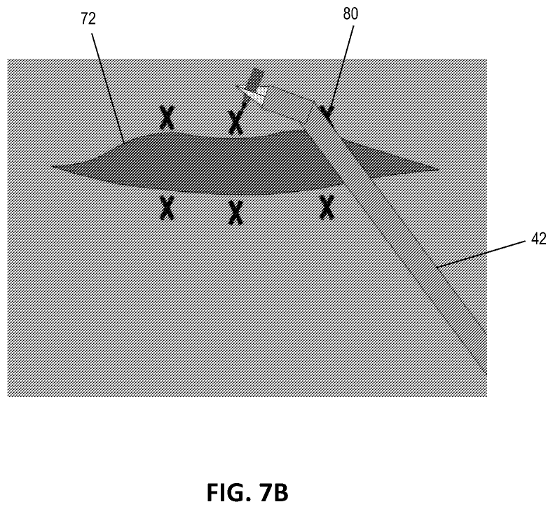

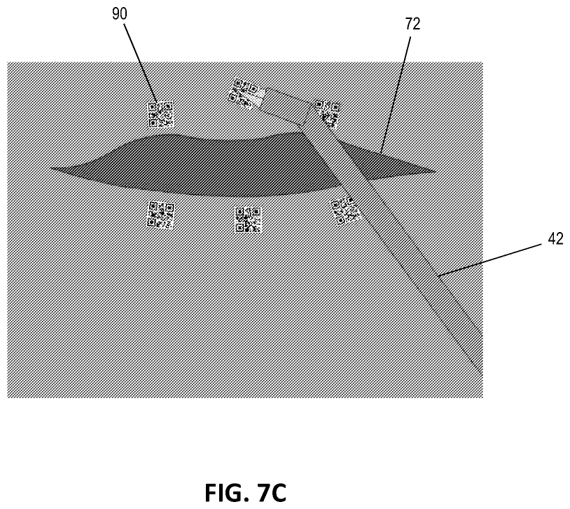

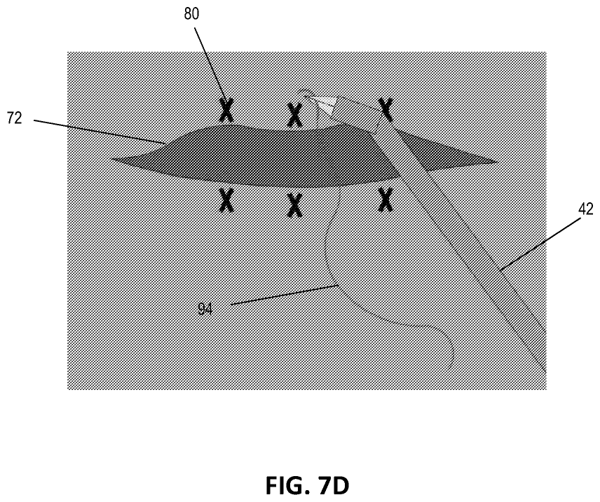

The markers can also be used, for example, to mark or identify where a selected surgical procedure or task, such as for example a suturing procedure, is to be performed. For example, one or more of the robotic arms 42 can be used by the surgeon to place a marker at a selected location, such as at or about an incision 72. As shown for example in FIG. 7A, the surgeon can control the robotic arm 42 to draw or place a marker 70 about or around the incision 72 in the patient's abdomen using for example a biocompatible pen with fluorescent dye or other imaging agent to mark the area to be sutured. The robotic arm 42 can also be employed to place a different type of marker, such as a series of “X” type markings, as illustrated in FIG. 7B. According to another practice, the surgeon can employ active or passive type markers. As shown for example in FIG. 7C, the robotic arm 42 of the robotic unit 50 can be employed to place QR code type markings 90 about the incision 72.

Once the markers have been placed at the selected surgical location, then the robotic unit 50 can be employed to perform the selected surgical task. For example, as shown in FIG. 7D, the robotic arm 42 can be controlled by the surgeon to place one or more sutures at the incision 72. The surgeon can, for example, place the suture using suitable biocompatible thread 94 at one or more of the markers, such as for example at the X shaped markings 80.

Alternatively, the controller 18, based on the image data 48 and the output signals generated by the detection unit 60, can automatically control the movement of the robotic arms to perform the surgical task, such as for example to create the incision 72 or to suture closed the incision.

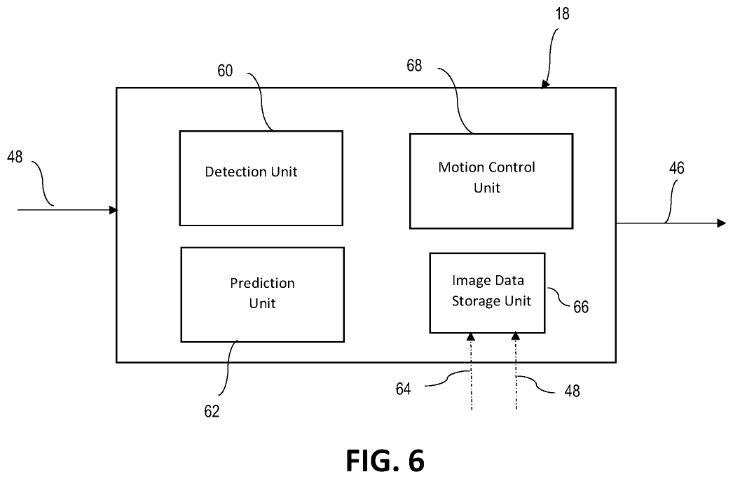

As shown in FIG. 6, the controller 18 may further include a detection unit 60 for detecting markers present in the image data 48 generated by the camera assembly 44. The controller 18 may also include a prediction unit 62 for analyzing the image data 48 to identify and/or predict selected types of images in the image data 48 by applying to the image data one or more known or custom artificial intelligence or machine learning (AI/ML) models or techniques. The prediction unit 62 can identify, based on the image data, selected markers or anatomical structure within the image data and can generate insights and predictions therefrom. According to one practice, the AI/ML techniques employed by the prediction unit 62 can be a supervised learning technique (e.g., regression or classified techniques), an unsupervised learning technique (e.g., mining techniques, clustering techniques, and recommendation system techniques), a semi-supervised technique, a self-learning technique, or a reinforcement learning technique. Examples of suitable machine language techniques include Random Forest, neural network, clustering, XGBoost, bootstrap XGBoost, Deep learning Neural Nets, Decision Trees, regression Trees, and the like. The machine learning algorithms may also extend from the use of a single algorithm to the use of a combination of algorithms (e.g., ensemble methodology), and may use some of the existing methods of boosting the algorithmic learning, bagging of results to enhance learning, incorporate stochastic and deterministic approaches, and the like to ensure that the machine learning is comprehensive and complete. The prediction unit 62 can be used to repeatedly label portions of the image data generated by the camera assembly 44 that correspond to regions of interest, such as markers or specific anatomical structure, such as tissue, veins, organs and the like. Further, the prediction unit 62 can be trained on sets of training data to identify the markers employed by the surgeon or selected anatomical structures of the patient. The illustrated controller 18 may also include an image data storage unit 66 for storing the image data 48 generated by the camera assembly 44 or image data 64 provided by a separate external data source. The external image data can include magnetic resonance imaging (MRI) data, X-ray data, and the like. The image data storage unit 66 can form part of the storage unit 24 or can be separate therefrom.

The controller 18 may also be configured to include a motion controller 68 for controlling movement of the robotic unit, such as for example by controlling or adjusting movement of one or more the robotic arms. The motion control unit may be configured to adjust the movement of the robotic unit based on the markers detected in the image data and/or selected anatomical structure identified in the image data. The markers may be detected by the detection unit 60 and the anatomical structure can be identified by the prediction unit 62. As contemplated herein, the motion control unit may be configured to adjust movement of the robotic unit by varying or changing the speed of movement of one or more of the robotic arms, such as by increasing or decreasing the speed of movement. The motion control unit may also be configured to adjust movement of the robotic unit by varying or changing the torque of one or more of the robotic arms. The motion control unit may also be configured to constrain, limit, halt, or prevent movement of one or more of the robotic arms relative to one or more selected planes or one or more selected volumes.

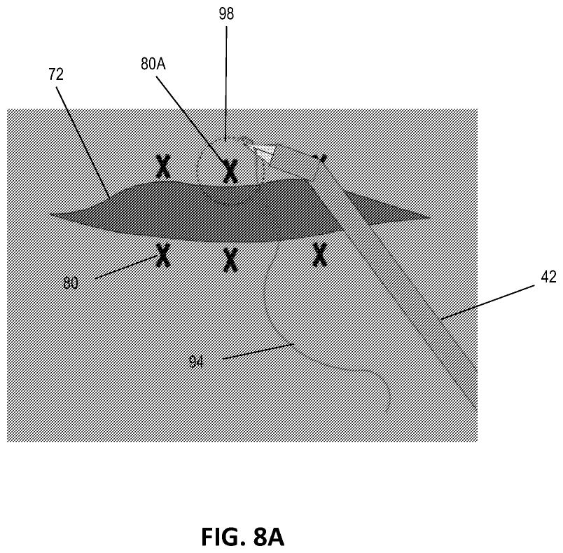

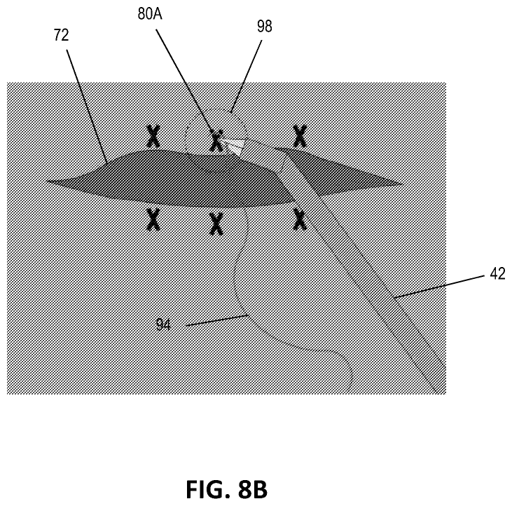

The surgical robotic system can also be configured to perform selected surgical tasks either manually (e.g., under full control of the surgeon), semi-autonomously (e.g., under partial manual control of the surgeon) or autonomously (e.g., fully automated surgical procedure). According to one practice of the present disclosure, as shown in FIG. 7D, the surgeon can place markers within the body of the patient, and then utilize the markers to guide the robotic unit under control of the surgeon to a selected surgical location to perform the surgical task, such as to throw one or more sutures, at the identified location. According to another practice, the system 10 can be configured to provide for semi-autonomous control, where the system 10 allows the surgeon to perform manual surgical tasks with a subsequent automated assist or control. For example, as shown in FIGS. 8A and 8B, the surgeon can apply the markers 80 about the incision 72 and then the surgeon can manipulate the robotic arm 42 to approach one of the markers 80A. The system 10 can be configured to store a preselected or predefined threshold distance 98 about the markers 80, such that when the end effector region of the robotic arm enters or falls within the threshold distance (e.g., less than the threshold distance), then the system automatically generates control signals 46 to operate the robotic arm 42 to automatically place or “snap” the end effector region directly to the location of the marker. Specifically, the image data 48 acquired by the camera assembly is processed by the detection unit 62 to identify the markers 80 and to detect the robotic arm location or proximity relative to the markers 80. The detected markings in the image data 48 can then be processed by the computing 18 and compared to the threshold distance 98. If the robotic arm 42 falls within the threshold distance from markers 80, such as for example the marker 80A, then the controller 18, via the motion controller 68, can generate control signals 46 that are processed by the robotic unit 50 to adjust the movement of the robot unit. According to one practice, the motion controller 68 via the control signals 46 adjusts the motion of the robotic unit, such as by increasing the speed of movement of either or both of the robotic arms, such that the robotic arm appears to “snap” to a location such that the end effector region of the robotic arm 42 is disposed immediately adjacent to the marker 80A. The automated placement of the robotic arm directly or immediately adjacent to the marker 80A ensures that the robotic arm is precisely located each time by the system 10. The surgeon can then manually throw the stitch or suture. The threshold distance can be stored at any suitable location in the system 10, and is preferably stored in the controller 18, such as in the storage unit 24 or in the motion controller 68.

Alternatively, the surgical robotic system 10 may be operated in a fully automated mode where the surgeon places the markers at selected locations within the patient with the robotic unit. Once the markers are placed into position, the system can be configured to perform the predetermined surgical task. In this mode, the image data 48 acquired by the camera assembly 48 can be processed by the detection unit 60 to detect the markers. Once the markers are detected, the motion controller 68, or alternatively the controller, may be configured to generate the control signals 46 for controlling or adjusting movement of the robotic unit 50 and for automatically performing with the robotic unit the selected surgical task. This process allows the surgeon to plan out the surgical procedure ahead of time and increases the probability of the robot accurately following through with the surgical plan in any of the autonomous, semi-autonomous and manual control modes. The various operating modes of the system 10 effectively allows the surgeon to remain in control (i.e. decision making and procedure planning) of the robotic unit while concomitantly maximizing the benefits of automated movement of the robotic unit.

The present disclosure also contemplates the surgeon utilizing the robotic arms 42 to touch or contact selected points within the patient, and the information associated with each contact location can be stored as a virtual marker. The virtual markers can be employed by the controller 18 when controlling movement of the robotic unit 50.

The surgical robotic system 10 of the present disclosure can also be configured to control or restrict the motion or movement of the robotic unit 50 when disposed within the patient. An advantage of restricting or limiting movement or motion of the robotic unit is minimizing the risk of accidental injury to the patient when operating the robotic unit, for example, during insertion of the robotic unit into a cavity, or moving the robotic unit within the abdomen of the patient, and swapping tools used by the robotic arms. To protect the patient from accidental and undetected off-camera injury, the system 10 may be configured to define a series of surgical zones, spaces or volumes in the surgical theater. The predefined zones may be used to constrain or limit movement of the robotic unit, and can also be used to alter, as needed, specific types of movement of the robotic unit, such as speed, torque, resolution of motion, volume limitations, and the like.

The present disclosure is directed to a system and method by which the surgical robotic system can aid the surgeon in performing the surgical task. The surgeon needs to be able to adapt to variations in the anatomy of the patient throughout the procedure. The anatomical variations can make it difficult for the system to adapt and to perform autonomous actions. The prediction unit 62 can be employed to enable the surgeon to address the anatomical variations of the patient. The prediction unit can identify from the image data selected anatomical structures. The data associated with the identified anatomical structures can be employed by the controller to control movement of the robotic unit.

As further shown in FIG. 6, the illustrated controller 18 can employ an image storage data unit 66 for storing image data associated with the patient, as well as related image data. The image data can include image data acquired by the camera assembly 44 as well as image data associated with other patient and acquired by other types of data acquisition techniques. For example, the patient image data acquisition techniques can include prestored image data associated with the patient, three-dimensional (3D) map information associated with the patient and the surgical environment or theater, as well as MRI data, X-ray data, computed tomography (CT) data, and the like. The 3D map can be generated from a variety of different data generation techniques known in the art, including light detection and ranging (LIDAR) techniques, stereoscopy techniques, image disparity techniques, computer vision techniques, and pre-operative or concurrent 3D imagery techniques. The prediction unit 62 can be employed to process and analyze the image data 48, and optionally process the image data stored in the image data storage unit 66, in order to automatically identify selected types of anatomical structures, such as organs, veins, tissue, and the like, that need to be protected from the robotic unit 50 during use. The prediction unit 62 can thus be configured or programmed (e.g., trained) to automatically identify the anatomical structures and to define, in combination with the motion controller 68, the types of motion controls to implement during the procedure. The present disclosure also contemplates the surgeon defining, prior to surgery, the types of motion limitations to implement during the surgical procedure.

As noted herein, during surgery, the surgeon frequently needs to adapt to variations in the anatomical structure of the patient. The anatomical variations of the patient oftentimes makes it difficult for the system 10 to properly function in semi-autonomous and autonomous operational modes, and also makes it difficult to prevent accidental injury to the patient when operating the robotic unit in manual mode. As such, in order to improve the overall efficacy of the surgical procedure and for the system 10 to reliably operate, the system can be configured to identify selected anatomical structure and then control or limit movement of the robotic unit during the surgical procedure based on the identified structure. The camera assembly 44 can be employed to capture image data of the interior of the abdomen of the patient to identify the selected anatomical structures.

According to the present disclosure, the motion controller 68 can generate and implement multiple different types of motion controls. According to one embodiment, the motion controller 68 can limit movement of the robotic unit to within a selected plane, within a selected volume or space, while also selectively limiting one or more motion parameters of the robotic unit based on a selected patient volume or space, proximity to the selected anatomical structures, and the like. The motion parameters can include range of motion, speed of movement in selected directions, torque, and the like. The motion controller 68 can also exclude the robotic unit from entering a predefined volume or space. The motion limitations can be predefined and pre-established or can be generated and varied in real time based on the image data acquired by the camera assembly during the surgical procedure.

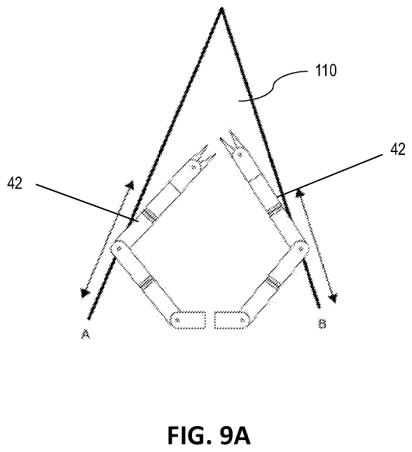

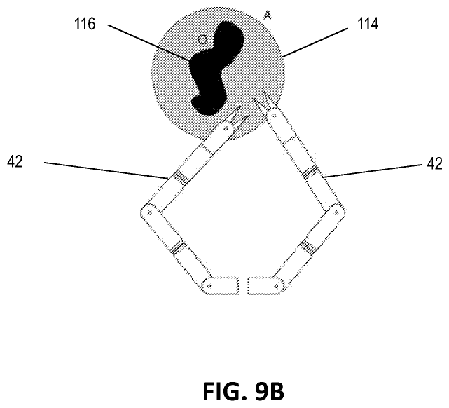

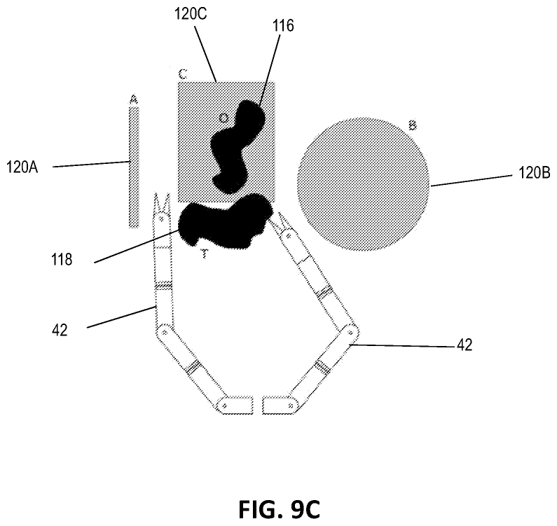

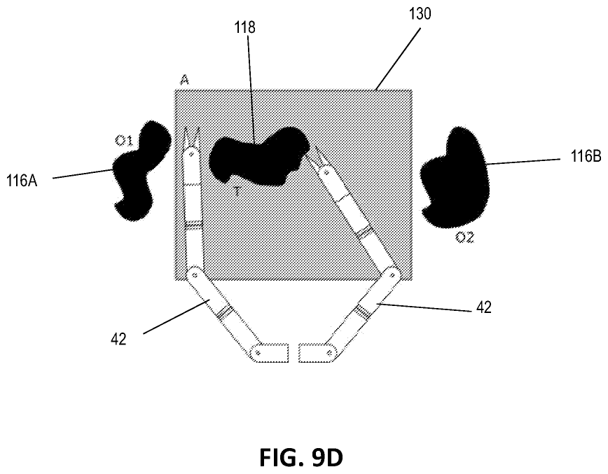

According to one embodiment, the controller 18 can define, based on the image data, a constriction plane for limiting movement of the robotic unit to within the defined plane. As shown for example in FIG. 9A, the controller 18 or the motion controller 68 can be employed to define a selected constriction plane 110 within the internal volume of the patient based on the image data 44. The robotic unit 50, and specifically the robotic arms 42, can be confined to movement within the constriction plane 110. Thus, even if the surgeon accidentally or purposely tries to move the robotic arms 42 to areas outside of the constriction plane 110, the motion controller 68 prevents this type of movement from occurring.

The motion controller 68 may also be configured to define a constraint volume, based on the image data and based on the output of the prediction unit 62, that constrains or limits movement of the robotic unit when positioned within the specified volume. The prediction unit 62 can be configured to receive and process the image data 48, and optionally the external image data 64, to identify or predict selected types of anatomical structures, such organs. The predicted or identified data associated with the anatomical structure can then be processed by the motion controller 68 to define a selected constraint volume about the anatomical structures or about a selected surgical site. According to one embodiment, as shown for example in FIG. 9B, the prediction unit 62 identifies the organ 116, and the motion controller 68 defines or generates a constraint volume 114 about the organ 116. When the robotic unit is positioned outside of or external to the constraint volume, then the motion controller 68 does not impose motion limitations on the robotic arms. However, when the robotic arms 42 enter the constraint volume 114, as shown, the motion controller 68 limits selected types of movement of the robotic arms. According to one example, the speed of movement of the robotic arms 42 is reduced by a selected predetermined amount. The speed reduction of the robotic arms provides an indication to the surgeon of approaching the organ. Those of ordinary skill in the art will readily recognize that other types of motion limitations can also be employed.

According to other embodiments, the controller 18 or the motion controller 68 can be configured to exclude the robotic unit from entering a defined space or eliminate or significantly reduce the motion capabilities of the robotic unit when in the defined space or volume. The prediction unit 62 can be configured to receive and process the image data 48, and optionally the external image data 64, to identify or predict selected types of anatomical structures, such organs or tissue. The predicted or identified anatomical structure data, such as the data associated with the organ 116, can be processed by the motion controller 68 to define a selected exclusion volume 120C about the organ 116. As shown for example in FIG. 9C, the motion controller 68 can also define additional exclusion zones or volumes, including the exclusion volumes 120A and 120B, which can define other areas of the patient volume that need or should be protected. The robotic arms 42 may be operated by the surgeon to perform a selected surgical task at the illustrated surgical site 118. The surgical site 118 can, by simple way of example, represent a tear that needs to be surgically closed. According to one example, the exclusion volumes 120A-120C can correspond to volumes that the robotic unit is prohibited from entering or penetrating, thus actively limiting the range of motion of the robotic unit and protecting the contents of the volume. According to one practice of the present disclosure, the motion controller 68 can be preconfigured to define one or more specific exclusion zones or volumes to protect a vital organ or tissue that should not be contacted.

According to some embodiments, the motion controller 68 may be configured to limit the extent or range of motion of the robotic unit to be within a specified volume or zone. In some applications, instead of defining multiple exclusion volumes or zones, the surgeon can instead define an inclusion volume, within which the robotic unit 50 is able to move freely. In the inclusion zone, the outside or external circumference or perimeter cannot be penetrated by the robotic unit. The prediction unit 62 can be configured to receive and process the image data 48, and in some embodiments the external image data 64, to identify or predict selected types of anatomical structures, such the organs 116A and 116B illustrated in FIG. 9D. The predicted or identified anatomical structure data, such as the data associated with the organs 116A, 116B, can be processed by the motion controller 68 to define a selected inclusion volume 130. The inclusion volume 130 can include, for example, the surgical site 118. The inclusion volume 130 can be configured to encompass the surgical site 118 while concomitantly avoiding or excluding the organs 116A and 116B, thus protecting the organs. The robotic arms 42 can be controlled by the surgeon to perform a selected surgical task at the illustrated surgical site 118 within the inclusion volume 130. While in the inclusion volume 130, the motion controller 68 is not configured to limit or constrain movement of the robotic unit, and as such the surgeon is free to control the robotic unit within the inclusion volume 130 without artificial limitations on speed and range of motion.

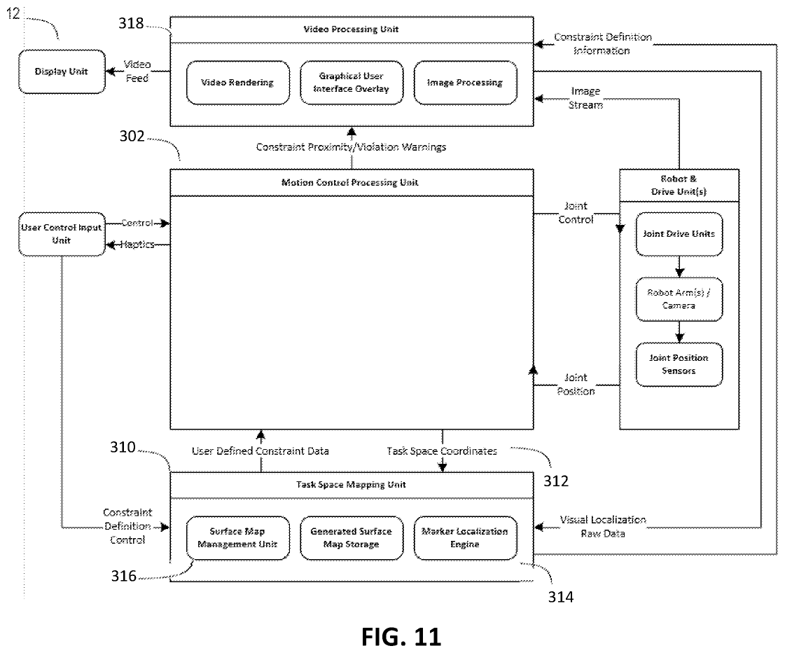

FIG. 11 schematically depicts an illustrative motion control system of a surgical robotic system, according to the teachings of the present disclosure. In some embodiments, control originates with a user interacting with positional control inputs, for example a hand controller, to provide task space positional commands to the Motion Control Processing Unit 302. The Motion Control Processing Unit 302 is configured to or programmed to generate individual joint position commands to achieve task space end effector position. The Motion Control Processing Unit 302 can include a combination of circuits and software to process the inputs and provide the described outputs.

The Motion Control Processing Unit 302 also provides logic to select an optimal solution for all joints within the residual degrees of freedom. In systems with more than 6 degrees of freedom supporting end effector position control, some joint positions are not discrete values, but a range of possible values throughout the range of residual degrees of freedom. Once optimized, joint commands are executed by the Motion Control Processing Unit 302. Joint position feedback comes back into the Motion Control Processing Unit 302 for determining end effector position error in task space after passing through forward kinematics processing.

A separate Task Space Mapping Unit 310 is depicted to describe the behavior of capturing constraint surfaces. In some embodiments, the Task Space Mapping Unit 310 is part of the Motion Control Processing Unit 302. Task Space Coordinates 312 of end effectors are provided to the mapping unit for creation and storage of task space constraint surfaces or areas. A Marker Localization Engine 314 is included to support changes to marker location driven by system configuration changes (e.g. burping the trocar), changes to visual marker location (e.g. as a result of patient movement), or in response to location changes of any other type of supported marker. A Surface Map Management Unit 316 supports user interaction with the mapping function to acknowledge updated constraint points or resolved surfaces.

During creation, or modification, or motion performance modification in response to, or motion violation of task space constraints, the Video Processing Unit 318 overlays pertinent information on a live image stream that can be ultimately rendered as video before being presented to the user on the Display Unit 12. Task space constraints may include a tissue surface (e.g. a visceral floor) and/or depth allowance, both of which are discussed in further detail below.

Once any part of the system operating within the abdominal cavity breaches the upper bound of depth allowance below, for example, the visceral floor surface, motion of that particular portion of the system can be disallowed. This may inherently cause all motion to be prevented. Once that occurs, there is an override functionality described which ensure users are not held captive.

In some embodiments, the system is employed in a patient's abdominal space, for example the area near the bowels. Whereas surface adhesions of bowel to other tissues can be visualized, manipulated, and then surgically addressed as part of a procedural workflow, tissues deeper within the viscera have both normal connective tissues and/or potentially unanticipated adhesions which cannot be easily visualized. Forcibly displacing tissue where attachments provide reactive forces to resist can quickly lead to trauma. When system 10 components operate without visualization at greater depths below the visceral floor, concern of lateral tissue movement causing trauma increases.

In abdominal surgeries insufflation provides intra-abdominal space above the viscera, thus creating the visceral floor. Aside from the benefit of enabling more space for visualization and access, the visceral floor is a somewhat arbitrary surface of interest. In ventral hernia repairs, there is often a hernia sac sitting outside the abdominal cavity protruding through the hernia itself. Prior to reduction of the contents of a hernia, there will be a column of bowel and adipose tissue rising up from the visceral floor to the hernia in the abdominal wall. In that scenario it is useful to establish a circumferential surface enclosing the tissue column to protect it from inadvertent and/or non-visualized contact.

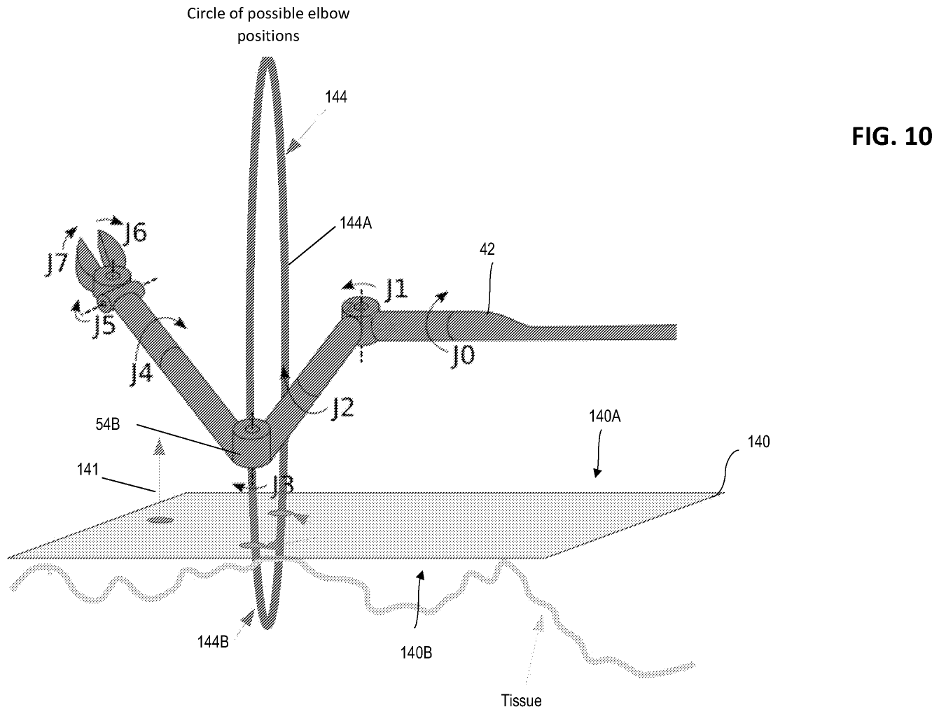

The system 10 can employ the controller 18 to define areas or zones of movement of the robotic unit, and conversely to define areas or zones where movement is restricted. According to some embodiments, as shown for example in FIG. 10, the controller 18 can be configured to define tissue contact constraint data, for example a two-dimensional model such as a constriction plane 140, that corresponds to the location of one or more selected anatomical structures of the patient, such as for example tissue, to be protected. The constriction plane 140 may be defined with a curvature.

In some embodiments, the controller may define a three dimensional area or volume rather than a plane. The volume may be shaped as a cube, cone, cylinder, or other useful three-dimensional shape.

In some embodiments, the tissue constraint data includes predetermined three dimensional or two dimensional shapes associated with a surgical area, for example an insufflated abdomen or chest cavity. In this way the robotic system may have a predefined constriction area to begin working with and can be updated to reflect the particular anatomy of a patient. In some embodiments, the tissue constraint data is calculated using markers, either virtual or physical, or by identifying portions of a tissue as discussed herein with regards to constriction areas or planes. In some embodiments, the predetermined tissue constraint data may be updated based on image data of a patient's surgical area or tissue identified within the surgical area.

The constriction plane 140 may lie directly on a physical tissue. For example, the constriction plane 140 may correspond to a defined, floor, for example a visceral floor. In some embodiments, the plane 140 may be at a specified distance above or below the tissue.

In some embodiments, surfaces of interest may be segmented by their sensitivity to contact. For example, liver tissue residing within the viscera may be separately identified. Liver tissue is soft and friable making it particularly sensitive to contact and creating a potential safety risk if damaged during surgery.

The user may identify the constriction plane 140 with a first robotic arm before insertion of subsequent robotic arms. The insertion of the second robotic arm may be monitored by the camera assembly 44, leaving the first robotic arm off-screen. Because the constriction plane 140 is already defined, the user can be alerted if the offscreen robotic arm dips into the plane 140.

The controller 18 may control the robotic arms 42 in a manner to reduce possible damage to tissue in an area identified by the tissue contact constraint data. The controller 18 is configured to or programmed to determine, relative to the constriction plane 140, the areas or zones that are safe to operate the robotic arms 42 of the robotic unit. For example, the controller 18 can be configured to or programmed to allow or permit movement of the robotic arms 42 on a first side 140A of the constriction plane 140 and to prohibit or constrict movement of the robotic arms on a second opposed side 140B of the plane. The second side 140B corresponds to an area of patient tissue of concern.

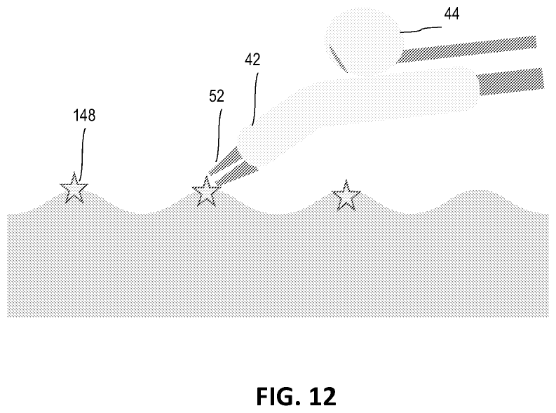

In some embodiments, the controller 18 is configured to or programmed to determine, relative to the constriction plane 140, a depth allowance up to which the robotic arms 42 can safely operate. The depth allowance is discussed in further detail below with regards to FIG. 12.

As shown in FIG. 10, the constriction plane 140 can include a point with a vector 141 normal to the intended plane in Cartesian coordinates. The normal vector 141 can be configured to point to the side of the constriction plane 140 where the elbow region 54B of the robotic arm is allowed to travel. As the placement of the elbow region is calculated, it is adjusted away from the prohibited side of the constriction plane 140.

The elbow region 54B of the robotic arm 42 can be moved, according to one movement sequence, in a circular motion as shown by the motion circle 144. Once the virtual constriction plane 140 is defined by the controller 18, and which corresponds to the anatomical structure that needs to be protected, the elbow region 54B of the robotic arm 42 can be permitted to move if desired along a first arc portion 144A of the motion circle 144 that is located on the first side 140A of the constriction plane 140. This first arc portion 144A may be referred to as the safe direction of movement. For example, the controller calculates the safe direction as “up” or away from gravity. In some embodiments, the elbow region 54B is prohibited from moving along a second arc portion 144B of the motion circle 44 that is located on the second side 140B of the constriction plane 140, so as to avoid contact with the tissue. By prohibiting movement of the robotic arm, such as the elbow region 54B on the second side of the constriction plane 140, the tissue of the patient is protected from accidental injury. Notably, multiple constriction planes may be combined to approximate more complex shapes.

In some embodiments, the user may redefine the constriction plane 140 after insertion of each robotic arm. However, immediately after insertion is completed, users may be required to establish the visceral floor surface and depth allowance before being able to freely operate the system 10 or be confronted with indications that they are proceeding at their own risk.

In order to prevent unacceptable non-visualized tissue contact by system 10 components, a user may define a boundary in space where the acceptability of incidental tissue contact begins to change to unacceptable contact. For hernia repair procedures with insufflated abdomens there is no specific point, line, or plane which defines this boundary, but a continuous planar surface approximating the visceral floor provides a useful model to control risk.

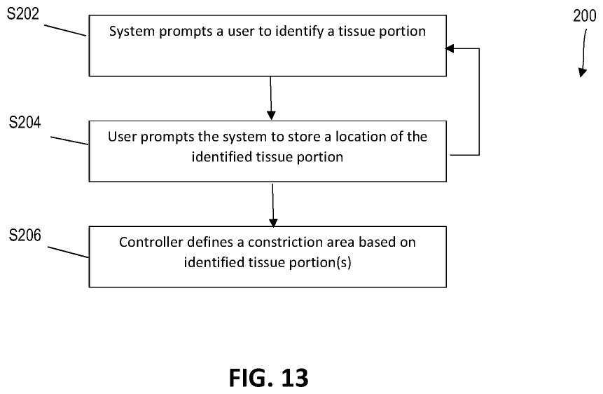

FIG. 12 is a representation of a tissue area identified by contact with a robotic arm, according to the teachings of the present disclosure. FIG. 13 is a flowchart representing a process 200 for identifying a tissue area. At step S202, the system 10 may prompt a user to identify a portion of tissue 148, for example by placing an end effector 52, or other distal end, of a robotic arm 42 into contact with the portion 148. For example, a user may touch the highest point within the abdomen to define a surface, for example a visceral floor. In some embodiments, the user need not physically touch a tissue, but may point at the portion 148 with the distal end of the robotic arm 42. In some embodiments, the robotic arm 42 includes one or more tissue contact sensors at a distal end of the arm. The tissue contact sensors may be shaped to reduce damage to a tissue when contacting the tissue. Force sensors could also be included in the robotic arms 42 to measure unintended forces acting on the arms by the contacted tissue.

Step S202 may be repeated one, two, or more times to identify multiple portions 148 of a tissue.

In an alternative embodiment, the tissue area may be identified using a single point laser range finder to define a horizontal plane. As another alternative, the tissue area may be identified using of a single visual marker and calibrated optics to use a focus position for range finding a point at which to create a horizontal plane. As another alternative, the tissue area may be identified by a manual angle definition around and relative to a gravity vector. An alternative embodiment involves the use of calibrated targets and optics to use a focus position for range finding of multiple visual targets. An alternative embodiment involves the use of integrated tissue contact sensors built into the instruments to define one or more points as described previously.

In some embodiments, two portions 148 of a tissue are identified by a robotic arm 42. Both points may lie on a defined constriction plane allowing for the inclusion of an angle. Rotation of the constriction plane around the line formed between the two portions 148 is further constrained by the gravity vector. Rotation of the constriction plane around the line used to define the plane is controlled by a secondary plane formed by the two portions on the line and the gravity vector. The constriction plane and the secondary plane must be perpendicular.

An alternative embodiment involves the use of a single visual marker of known shape and dimensions to estimate position and orientation based on images of the marker by a single imager camera system with known optical properties. One example is an equilateral triangle cut from a thin but stiff material. Placing the rigid shape on top of tissue aligns the shape with the tissue plane. Imaging the shape from a known position will cause some degree of size variation and distortion. Given optics with known distortion characteristics, image data can be processed to infer the distance and orientation of the visual marker. This same approach could be used with a dual imager system and improved by leveraging visual disparity.

The method continues at step S204, when the user prompts the system 10, for example by pressing a button on the hand controller 17 or foot pedal assembly 19, manipulating a grasper of a robotic arm 42, or giving a vocal cue, to store a location of the identified portion(s) of tissue 148 for the purposes of defining tissue area. The location may be stored in a memory of the controller 18. In some embodiments, the user identifies multiple portions of tissue 148, for example with a robotic arm 42, before a tissue area is defined. The user may prompt the system 10 after identifying each portion 148 or may prompt the system after identifying multiple portions in succession.

At step S206, the controller defines a constriction area based on the one or more identified portions of tissue 148. As described above, the constriction area may be a three-dimensional volume or a two-dimensional plane. For example, the controller may define a plane representative of the visceral floor. In some embodiments, the controller defines tissue contact constraint data based on the one or more identified portions of tissue 148. The tissue contact constraint data may include a constriction area or plane, or may include a predefined volume associated with a tissue site.

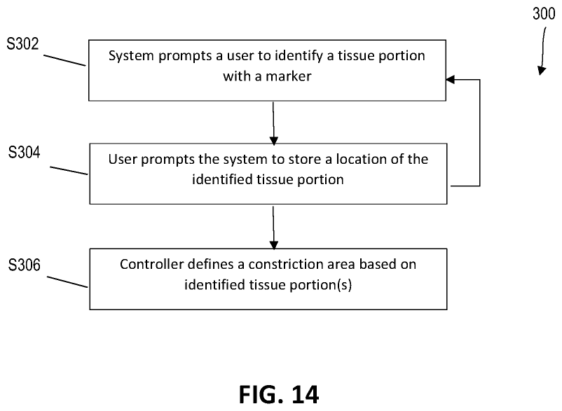

FIG. 14 is a flowchart representing a process 300 for identifying a tissue area. At step S302, the system 10 may prompt a user to identify a portion of tissue 148, for example by identifying a marker placed on the portion 148. The marker may be any marker as described herein above. Step S302 may be repeated one, two, or more times to identify multiple portions 148 of a tissue. Alternatively, multiple portions 148 of a tissue may be marked with a marker, and the multiple markers may be identified at once.

The method continues at step S304, when the user prompts the system 10, for example by pressing a button on the hand controller 17 or foot pedal assembly 19, manipulating a grasper of a robotic arm 42, or giving a vocal cue, to store a location of the identified portion(s) of tissue 148 for the purposes of defining tissue area. The location may be stored in a memory of the controller 18.

At step S306, the controller defines a constriction area based on the one or more identified portions of tissue 148. As described above, the constriction area may be a three-dimensional volume or a two-dimensional plane. For example, the controller may define a plane representative of the visceral floor.

In some embodiments, the system 10 projects an image of the constriction area on top of an existing video feed provided to a user for the purpose of evaluation or confirmation.

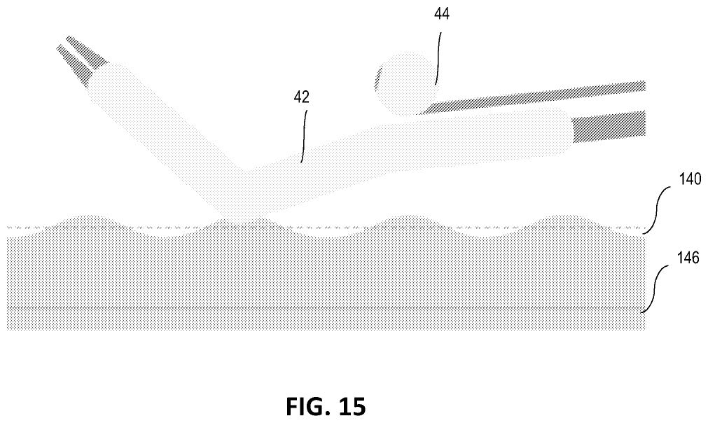

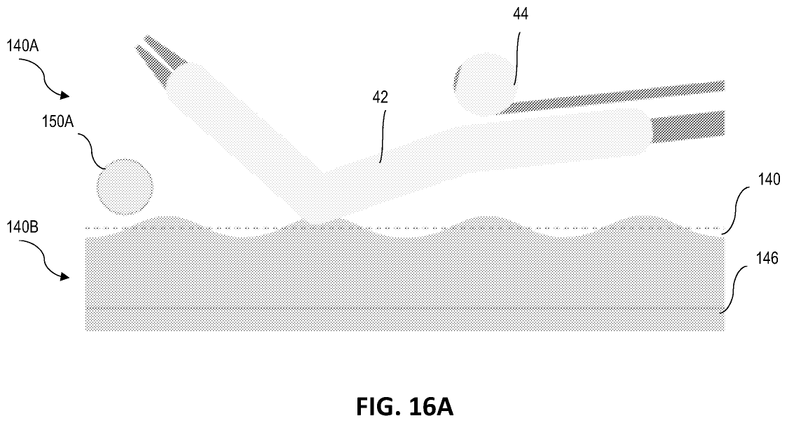

The following example uses a defined visceral floor but other anatomical elements are equally compatible where the system 10 defines a plane or area, and it is desirable to define a depth allowance beyond the defined plane or area. In some embodiments, the depth allowance defines a tissue depth relative to a floor in which one or more constraints may be applied to control the robotic arms between the floor and the depth allowance. FIG. 15 is a representation of a defined depth allowance below a visceral floor, according to the teachings of the present disclosure. Potential for risky non-visualized tissue contact increases with depth below a surface approximating the visceral floor. Visceral tissues tend to roughly self-level under the influence of gravity, but not perfectly; mounding, slanting, or cupping are possible. The term “below” when referring to the visceral floor surface refers to the normal direction relative to the visceral floor surface regardless of patient or system 10 orientation, pointing into the viscera. Specific sensitivity to the degree of non-visualized tissue contact from system 10 components travelling below the visceral floor is unique to each particular patient and is informed by the user's medical expertise and training.

In some embodiments, the controller 18 user control of the arms 42 is reduced as the arms 42 move past a defined visceral floor, constriction area, constriction plane, or other defined constraint. For example, the controller 18 may increase constraints on speed of movement of the arms 42 as the arms 42 more past the defined visceral floor, constriction area, constriction plane, or other defined constraint. Additionally or alternatively, the controller 18 may increasingly reduce the torque of the arms 42 as the arms 42 more past the defined visceral floor, constriction area, constriction plane, or other defined constraint.

The system 10 may also provide sensory feedback to a user when one or more arms 42 reach or cross the defined visceral floor. Sensory feedback may include visual indicators on a display, an audio cue or alarm (i.e., a ring, bell, alarm, or spoken cue), and/or haptic, tactile feedback to the hand controllers. Similar or different sensory feedback may be provided if one of the arms 42 reaches or crosses a defined depth allowance.

In some embodiments, the controller 18 may be configured or programmed with a predetermined depth allowance at a specified distance below the constriction area or plane 140, for example a defined visceral floor. In some embodiments, the memory holds executable depth allowance instructions to define a depth allowance relative to the constriction area. In some embodiments, users may determine the appropriate depth allowance 146 below a defined visceral floor. In some embodiments, setting the depth allowance 146 involves use of a slider switch or one or more up/down button presses to navigate a list of pre-programmed depth increments. Based on the patient's habitus, the user may decide to adjust the depth allowance 146 from its default value. For example, patients with higher BMI may have a thicker layer of fatty tissue at the top of the viscera, so the user may increase the depth allowance 146 to account for the added padding between the top plane and more delicate structures.

The controller 18 may be configured to or programmed with a default upper limit of travel depth allowance to remove the potential for misuse where unreasonable travel depth allowance values can be chosen. For example, allowing a depth allowance of 1 meter would be unacceptable and serve to override the protection provided. In some embodiments, the upper limit of travel depth allowance is set at 2 centimeters to ensure a reasonable maximum travel limit below the visceral floor surface where incidental contact will not lead to unacceptable risk of harm to patients. The upper limit may be 0, 1, 2, 3, 4, 5, 6, 7, 8, 9, or 10 centimeters, or any distance there between. In some embodiments, the depth allowance may be a negative value such that the depth allowance is “above” the constriction area 140. For example, the upper limit may be −1, −2, −3, −4, −5, −6, −7, −8, −9, or −10 centimeters, or any distance there between.

In some embodiments, the user selects a depth allowance by engaging a slider control input, for example on the hand controller. In an alternative embodiment, the user may move an end effector away from a defined constriction area or surface at a distance that will used as the depth allowance. In another alternative embodiment, the user may select from a pre-existing set of standard depth allowances based on the location of the surface constraint, patient orientation, the region of the abdomen in which the procedure is focusing, or any such similar anatomically driven preset definition.

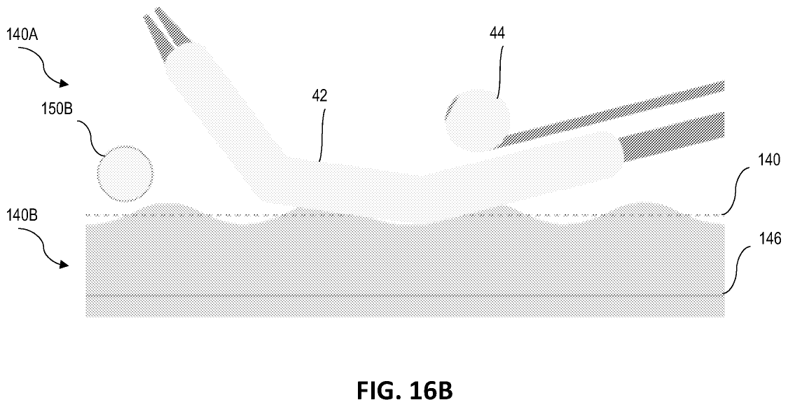

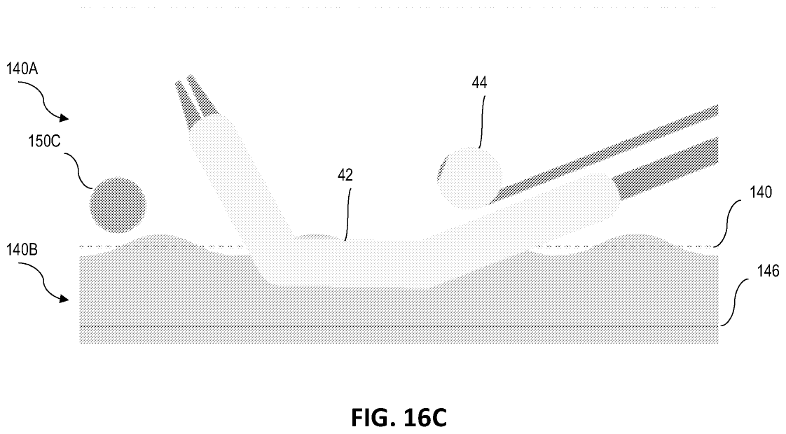

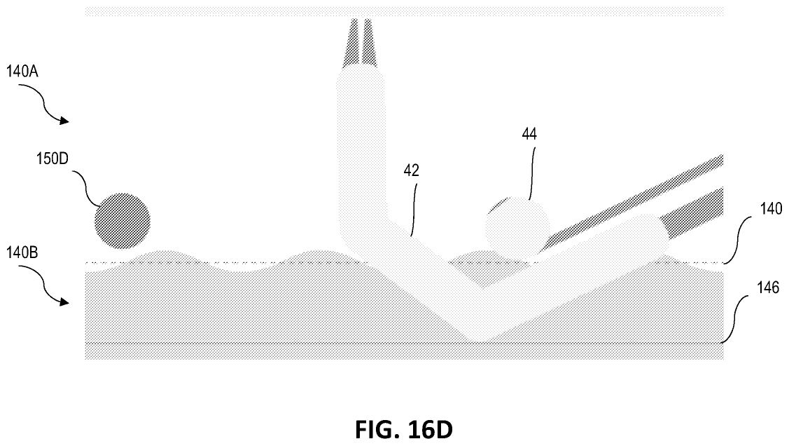

In addition, or in the alternative, to prohibiting or constricting movement of the robotic arms 42 on a second opposed side 140B of the plane, the system may provide one or more warnings to a user that a robotic arm 42 is approaching or has entered a plane 140. For example as shown in FIG. 16A, if the robotic arm 42 is more than a predetermined distance from the plane 140, the system 10 may provide, for example on a display, a safe indication 150A to the user that the arm 42 is in a “safe” area relative to the plane 140. A safe indication 150A may include, for example, a green light. In some embodiments, the system 10 provides no indication to a user when the robotic arm 42 is in a “safe” area.

In some embodiments, for example, as shown in FIG. 16B, the system 10 may provide a warning indication 150B to the user that the arm 42 is below the plane 140. A warning indication 150B may include, for example, a yellow light. The warning indication 150B may be triggered when the robotic arm 42 is below the plane 140, but above, for example, a midpoint between the plane 140 and a depth allowance 146. In some embodiments, the system may reduce the speed of movement of the robotic arms 42 when the robotic arm 42 is below the plane 140, but above a midpoint between the plane 140 and a depth allowance 146.

In some embodiments, for example, as shown in FIG. 16C, a further warning indication is provided to the user when the arm 42 operates at or within an approximate midpoint of the depth allowance 146 from the plane 140. The further warning indication 150C. The further warning indication 150C may include, for example, an orange light. In some embodiments, the system may further reduce the speed of movement of the robotic arms 42 when the arm 42 operates at or within an approximate midpoint of the depth allowance 146 from the plane 140

As depicted in FIG. 16D, the system 10 may provide a danger indication 150D to the user that the arm 42 is at or immediately adjacent to the depth allowance 146. A danger indication 150D may include, for example, a red light. As another example, the plane 140 may be a defined visceral floor as discussed above. The system 10 may provide a danger indication 150D if the robotic arm 42 is at or immediately adjacent to a depth allowance 146 defined by the user or system 10. In some embodiments, movement of the robotic arms 42 is prevented or halted below the depth allowance 146 when a danger indication 150D is provided.

In circumstances where the user determines a need to override the depth allowance due to an acute issue requiring intervention, the user may engage a manual override. During an override, existing status indications may not be disabled but may be modified to show that the system 10 is in an override condition. When an override is no longer needed, the user may not have to manually disengage the override. For example, if the user overrides the limit on operation below the depth allowance and then brings the arms back within the previously established depth allowance limit, the override may be automatically cancelled.

The indications discussed above may be provided in the form of tactile feedback. For example, one or more the hand controllers 17 may vibrate if one of the robotic arms 42 contacts the constriction plane 140, passes the constriction plane 140, or comes within a predetermined threshold of the constriction plane 140. The vibration may increase in strength as one of the arms 42 draws closer to the constriction plane 140.

The surgical robotic system 10 of the present disclosure can also be configured to control or restrict the motion or movement of the robotic unit 50 relative to a constriction area 140 or depth allowance 146. For example, the system 10 may prevent or halt the robotic arms from moving past the constriction area 140. In some embodiments, the system 10 allows movement of the arms 42 along a virtual constriction area 140, particularly if the area 140 is situated at a distance from tissue. The Motion Control Processing Unit may assign an increasing cost to a joint position as that particular joint operates closer to the depth allowance. This would provide preventative adjustment to reduce the utilized depth allowance 146.

A user may redefine an already established virtual constriction plane 140. For example, during operation the user may have made changes to the virtual center position (i.e. “burp” the trocar forming the patient port) which requires adjustments to the relative location of the user defined visceral floor surface. The relative position of the surface must be adjusted to account for the corresponding movement of the instruments and camera relative to the visceral floor. To do so, a user prompts the system 10, for example by pressing a button or giving a vocal cue, to define a new virtual constriction plane 140. The user may then proceed to define the new plane using markers or end effectors as described above. The plane 140 may need to be redefined if the patient moves or is moved, or if the robotic arms are situated in a new direction or in a new area. In some embodiments, the system 10 may automatically recalculate the plane 140 when the robotic arms are situated in a new direction or in a new area.

In alternative embodiments, the system 10 employs complex surface definition utilizing DICOM format CT or MRI images to define surface boundaries based on differences in tissue density and types. This type of information would likely need to be obtained from intra-operative imaging due to differences in insufflated abdomens. As another alternative, the system 10 may utilize the shape of the instrument arms themselves as placed and selected by the user to define a collection of lay-lines which are lofted together to define a boundary surface within which to operate. As another alternative, the system 10 uses visual disparity to generate or map a 3D point cloud at the surface of existing tissue. The use of Simultaneous Localization and Mapping (SLAM) algorithms to achieve this mapping is a well-known technique. As another alternative, the system 10 uses point or array LIDAR data accumulated over time to construct a surface map from range data relative to the system coordinate frame. As another alternative, the system 10 uses multiple visual markers of known shape and size placed at various locations on a tissue surface to determine distance, location, and orientation of points along that surface. This embodiment uses the same camera system characterization as the single visual marker embodiment for single plane definition.

In alternative embodiments, the system 10 employs customization of surface constraints at specific locations, which employs a user interface for selecting a local region of a constraint surface to define a smaller depth allowance than the rest of the constraint surface. As another alternative, the system 10 employs use of fluorescent dye and/or imaging to define areas of high perfusion where depth allowances are decreased.

In alternative embodiments, the system 10 uses visual markers to provide a dead reckoning sensing for a constraint surface plane. Monitoring the location of this dead reckoning visual marker will determine if the constraint surface has moved. As another alternative, the system 10 monitors insufflation pressure to determine when the viscera is likely to have moved. As another alternative, the system 10 uses a specific localization sensor placed on the patient's anatomy where constriction area is defined. As this localization sensor moves, so does the constriction area. Localization could be achieved many ways including electromagnetic pulse detection.

In an alternative embodiment, the system 10 employs sensor fusion of internal robotic control feedback (current monitoring, proximity sensor fusion, and the like) with proximity to constriction areas. Feedback from the system can be used to modify the interpretation of an operation relative to a constriction area.

In some embodiments, the controller 18 limits lateral (i.e. parallel to constriction surfaces) movement in proportion to the degree to which the robot or camera has intruded past the constriction area towards the depth allowance. In another alternative embodiment the controller 18 utilizes a task space cost function to minimize the amount of depth allowance utilized by any given joint.

The many features and advantages of the disclosure are apparent from the detailed specification, and thus, it is intended by the appended claims to cover all such features and advantages of the disclosure which fall within the true spirit and scope of the disclosure. Further, since numerous modifications and variations will readily occur to those skilled in the art, it is not desired to limit the disclosure to the exact construction and operation illustrated and described, and accordingly, all suitable modifications and equivalents may be resorted to, falling within the scope of the disclosure.

What is claimed is:

1. A surgical robotic system, comprising:
a robotic unit having robotic arms;
a camera assembly to generate a view an anatomical structure of a patient;
a memory holding executable instructions to control the robotic arms,
a controller configured to or programmed to execute instructions held in the memory to:
receive tissue contact constraint data; and
control the robotic arms in a manner to reduce possible damage to tissue in an area identified by the tissue contact constraint data; and
a display unit configured to display a view of the anatomical structure.

2. The surgical robotic system of claim 1, wherein the controller executes instructions held in memory to define a floor relative to the tissue based on the contract constraint data, the floor defines an area or region in which the robotic arms can operate with minimal risk to damage to the tissue.

3. The surgical robotic system of claim 2, wherein the controller executes instructions held in memory to define a depth allowance relative to the floor based on the tissue contract constraint data, the depth allowance defines a tissue depth relative to the floor in which one or more constraints may be applied to control the robotic arms when the robotic arms are between the floor and the depth allowance.

4. The surgical robotic system of claim 3,
wherein the memory holds executable instructions to halt movement of the robotic arms when one of the robotic arms goes past the depth allowance; and
wherein the controller executes the instructions to halt movement of the robotic arms past the depth allowance when the one of the robotic arms goes past the depth allowance.

5. The surgical robotic system of claim 1, wherein the controller is configured to or programmed to define a constriction area based on the tissue contact constraint data and a portion of an anatomical structure identified with a distal end of at least one of the robotic arms.

6. The surgical robotic system of claim 5, wherein the display unit is configured to display an indicator when one of the robotic arms enters a space beyond the constriction area.

7. The surgical robotic system of claim 5, wherein the memory holds executable depth allowance instructions to define a depth allowance relative to the constriction area.

8. The surgical robotic system of claim 7, further comprising a display unit, the display unit is configured to display an indicator when the one of the robotic arms enters an area between the constriction area and the depth allowance.

9. The surgical robotic system of claim 7, wherein the display unit is configured to display an indicator when the one of the robotic arms reaches the depth allowance.

10. The surgical robotic system of claim 5, wherein the controller is configured to or programmed to execute instructions held in the memory to limit movement of the robotic arms to within the constriction area.

11. The surgical robotic system of claim 7, wherein the controller is configured to or programmed to execute instructions held in the memory to halt movement of the robotic arms beyond a depth allowance defined relative to the constriction area.

12. The surgical robot system of claim 5, wherein the constriction area is represented by a plane and the controller is configured to programmed to halt movement of the robotic arms on one side of the plane.

13. A method for controlling a location of one or more robotic arms in a constrained space, comprising:
receiving tissue contact constraint data;
defining an area identified by the tissue contact constraint data; and
controlling the one or more robotic arms in a manner to reduce possible damage to tissue in the area defined by the tissue contact constraint data.

14. The method of claim 13, further comprising:
identifying a portion of an anatomical structure with a distal end of one of the robotic arms; and
defining a constriction area based on the tissue contact constraint data and the identified portion of an anatomical structure.

15. The method of claim 14, further comprising displaying, on a display unit, an indicator when one of the robotic arms enters a space beyond the constriction area.

16. The method of claim 13, further comprising defining a floor relative to the tissue in the area identified by the tissue contact constraint data, the floor defines an area or region in which the one or more robotic arms can operate with minimal risk to damage to the tissue.

17. The method of claim 16, further comprising defining a depth allowance relative to the floor based on the tissue contract constraint data, the depth allowance defines a tissue depth relative to the floor in which one or more constraints may be applied to control the one or more robotic arms when the robotic arms are between the floor and the depth allowance.

18. The method of claim 17, further comprising halting movement of the one or more robotic arms when the one or more robotic arms extend past the depth allowance.

19. The method of claim 17, further comprising displaying an indicator, on a display unit, when one of the robotic arms reaches the depth allowance.

20. The method of claim 17, further comprising slowing movement of the robotic arms when the robotic arms are between the depth allowance and the constriction area.

21. The method of claim 17, further comprising halting movement of the robotic arms when the robotic arms extend beyond the depth allowance.

22. The method of claim 13, wherein the constriction area is represented by a plane and controlling the one or more robotic arms in a manner to reduce possible damage to tissue in the area defined by the tissue contact constraint data comprises halting movement of the one or more robotic arms on one side of the plane.

23. A surgical robotic system, comprising:
a robotic arm assembly having robotic arms;
a camera assembly, wherein the camera assembly generates image data of an internal region of a patient, and
a controller configured to or programmed to:
detect one or more markers in the image data,
control movement of the robotic arms based on the one or more markers in the image data, and
store the image data.

24. The surgical robotic system of claim 23, wherein the controller is configured to or programmed to control the robotic arms to place the one or more markers.

25. The surgical robotic system of claim 23, wherein the markers include an X shape, quick response (QR) code markings, reflective tape, reflective film, stickers, cloth, staples, tacks, LED objects, emitters.

26. The surgical robotic system of claim 23, wherein the controller is configured to or programmed to define a threshold distance relative to the one or more markers, and vary the speed of movement of at least one of the robotic arms when one of the robotic arms is disposed relative to the one or more markers at a distance that is less than the threshold distance, such that one of the robotic arms is automatically placed adjacent to the one or more markers.
